(19) United States
(12) Patent Application Publication
(10) Pub. No.: US 2022/0064291 A1
(43) Pub. Date: Mar. 3, 2022

(54) METHODS OF USING BUTYROPHILIN ANTIBODIES FOR TREATING HIV INFECTION

(71) Applicant: Merck Sharp & Dohme Corp., Rahway, NJ (US)

(72) Inventors: Bonnie Jean Howell, Harleysville, PA (US); Hsien-Wei Yvonne Meng, Brookline, MA (US); Morgan Ann Monslow, Springfield, PA (US); Hussam Hisham Shaheen, Auburn, NH (US); Sai Vikram Vemula, Edison, NJ (US)

(73) Assignee: Merck Sharp & Dohme Corp., Rahway, NJ (US)

(21) Appl. No.: 17/415,031

(22) PCT Filed: Dec. 17, 2019
(86) PCT No.: PCT/US2019/066683

§ 371 Date: Jun. 17, 2021

Related U.S. Application Data
(60) Provisional application No. 62/781,204, filed on Dec. 18, 2018.

(51) Int. Cl. C07K 16/28

(52) U.S. Cl. CPC C07K 16/2803; C07K 2317/92; C07K 2317/76; C07K 2317/30

(57) ABSTRACT

The present invention relates to methods for treating individuals infected with the human immunodeficiency virus (HIV) comprising administering to the subject with an antibody to Butyrophilin that reactivates HIV from latency and/or activates CD4+ T cells.

### FIELD OF THE INVENTION

The present invention relates in part to a method of treating HIV infection by administering a butyrophilin antibody, optionally in combination with a latency reversing agent, or one or more anti-retroviral agent(s).

### BACKGROUND OF THE INVENTION

Human immunodeficiency virus (HIV) has been identified as the etiological agent responsible for acquired immune deficiency syndrome (AIDS), a fatal disease characterized by destruction of the immune system and the inability to fight life-threatening opportunistic infections. Highly active antiretroviral therapy (HAART) has been used to effectively suppress replication of HIV (Gulick et al. (1997) N. Engl. J. Med. 337:734-9; Hammer et al. (1997) N. Engl. J. Med. 337:725-733). However, HAART is primarily efficacious with regard to the prevention of the spread of infection into uninfected cells and this therapy does not eradicate the virus due to the integration of latent proviral DNA into the host cellular genome (Wong et al. (1997) Science 278:1291-1295; Finzi et al. (1997) Science 278:1295-1300 (see comments); Finzi et al. (1999) Nat. Med. 5:512-517; Zhang et al. (1999) N. Engl. J. Med. 340:1605-1613). HIV will remain a chronic viral infection unless there are therapeutic treatments for addressing viral persistence and the latently infected viral reservoir.

Butyrophilins (BTNs) are a novel class of immunomodulatory receptors (IMRs). BTNs share considerable structural homology with the B7 family of proteins (e.g, CD80, CD86, PD-L1, and PD-L2), including similar extracellular IgV and IgC domains. BTNs are primarily expressed on macrophages, dendritic cells, B cells, NK cells, and T cells and have also been reported to be upregulated in stressed and metastatic cells as well as several human cancers. BTNs can modulate T cell responses, regulating both α/β and γ/δ T cell function. Among BTN-like family members (BTNL2, BTNL3, BTNL8, BTNL9, BTNL10), only BTNL8 is believed to be immunostimulatory while all others are believed to be immunoinhibitory. Structural and sequence homology within the BTN family (BTN1A, BTN2A, BTN3A) is very high. Amino acid sequence homology between BTN3A isoforms is >90% and amino acid sequence homology between BTN3A and BTN2A is ˜45%. BTNs can be expressed as heterodimers. There is a need in the art to explore utilizing modulators to immunomodulatory receptors to further eradicate the HIV infection.

### SUMMARY OF THE INVENTION

The present invention relates to methods for treating HIV infection. The present invention is based, in part, on the discovery that butyrophillin (BTN) protein is enriched on HIV-1 infected T cells.

In one embodiment, BTN proteins suppress viral expression and thus antibodies against butyrophillin can be used to activate the expression of quiescent, integrated HIV within resting CD4⁺ T cells. Selective reactivation of latent infection may allow antiretroviral drugs and the antiviral immune response to recognize and clear residual HIV infected cells. Therefore, in one aspect, the invention relates to a method of treating HIV infection in a subject comprising administering an anti-BTN antibody that activates the expression of HIV within CD4+ T cells and optionally activates T cells. In another aspect, the invention relates to a method of treating HIV infection in a subject comprising administering an anti-BTN antibody that activates CD4+ T cells. In one embodiment, the anti-BTN antibody is an antagonist. The method may further comprise administering one or more anti-retroviral agents or therapeutic agents to enhance immune clearance or control residual viral reservoir. The method may further comprise administering a latency reversing agent, such as epigenetic modulators, molecules that alter cell metabolism and transcription, or cell signaling molecules, to enhance latency reversal. In one embodiment, the latency reversing agent is a histone deactylase inhibitor.

Other aspects of the invention relate to a method of clearing HIV infection with an effective amount of anti-BTN antibody. In one embodiment, the anti-BTN antibody kills the HIV infected T cells through immune-mediated effector function.

Suitable anti-retroviral agents for use in the therapeutic compositions and methods described herein include entry inhibitor, fusion inhibitor, integrase inhibitor, nucleoside reverse transcriptase inhibitors, non-nucleoside reverse transcriptase inhibitors, protease inhibitors, co-receptor antagonists, retroviral integrase inhibitors, viral adsorption inhibitors, viral specific transcription inhibitors, and cyclin dependent kinase inhibitors. In one embodiment, the anti-retroviral agent is selected from the group consisting of doravirine, islatravir, efavirenz, indinavir sulfate, and raltegravir potassium.

### BRIEF DESCRIPTION OF THE DRAWINGS

The foregoing and other objects, features and advantages of the invention will be apparent from the following more particular description of various embodiments of the invention, as illustrated in the accompanying drawings.

FIG. 1: Expression of butyrophilins on HIV-1 infected CD4+ T cells. Uninfected, active HIV-1 infected, and latent HIV-1 infected CD4+ T cells were stained with anti-BTNL8 (top) or anti-BTN2A2 (bottom) antibodies and protein expression was quantified by flow cytometry (APC stands for allophycocyanin and FITC stands for fluorescein).

FIG. 2: Enrichment in HIV DNA levels following pull down with antibody beads. HIV DNA levels in HIV infected CD4+ T cells from 4 donors were quantified after antibody pull down. Data is presented as HIV DNA copies/200,000 cells. Black bars=total CD4+ T cells; White bars=BTN3A antibody pulldown cells.

FIG. 3A-D: BTN modulation of T cell activation. Recombinant BTN proteins, BTN2A2 and BTN3A1, and PD-L1 protein block activation of human CD4+ T cells following stimulation with a human anti-CD3 antibody in both bead—(A-C) and plate-based assays (D).

FIG. 4: Anti-BTN3A antibody (clone 20.1) enhances T cell activation mediated by anti-CD3 antibody. Plates were coated with anti-CD3 antibody and varying concentrations of anti-BTN3A antibody (20.1). As controls, plates were coated with anti-CD3 antibody and varying concentrations of isotype control, anti-CD3 antibody alone, or isotype control alone.

FIG. 5: Anti-BTN3A antibody (clone 20.1) enhances virus reactivation mediated by anti-CD3 antibody in HIV latent primary CD4+ T cells. Plates were coated with anti-CD3 antibody and varying concentrations of anti-BTN3A antibody (20.1). As controls, plates were coated with anti-CD3 antibody and varying concentrations of isotype control, anti-CD3 antibody alone, or isotype control alone. Luminescence is measured by RLU.

FIG. 6: Recombinant BTN3A proteins block anti-BTN3A antibody (clone 20.1)-mediated virus reactivation in HIV latent primary CD4+ T cells. Plates were coated with anti-CD3 and anti-BTN3A antibody (20.1). BTN3A1-Fc or BTN3A2-Fc protein was added to block anti-BTN3A antibody-mediated virus reactivation. BTN2A2-Fc and IgG-Fc proteins were used as negative controls.

FIG. 7: Treatment of peripheral blood mononuclear cells (PBMC) with anti-BTN3A antibody (clone 103.2) increases IFNγ (left) and TNFα (right) cytokine production in HIV-positive (virally suppressed) and negative donors in the presence of HIV-gag pool-peptide stimulation. PBMC were pre-incubated with either no antibody, anti-BTN3A (20.1 or 103.2), anti-PD-1 (in-house MK3475), or isotype control antibody 30 minutes prior to adding HIV-gag pool-peptide stimulation. After 6 hours of culture, cytokine production was assessed in the supernatant. Data are normalized as fold-change relative to HIV-gag alone stimulated wells.

FIG. 8: BTN3A1 antibody campaign screening data. Each column depicts antibodies that fall within one of the three functional categories: Virus reactivation and T cell activation, T cell activation without virus reactivation, and virus reactivation without T cell activation. Viral reactivation (top row) is expressed as fold change over CD3 control and dotted line represents the 95th percentile cut point. IFN gamma expression (bottom row), indicative of T cell activation, is expressed as fold change over CD3 control and dotted line represents the 95th percentile cut point.

### DETAILED DESCRIPTION OF THE INVENTION

Methods and compositions are provided for treating patients infected with HIV. In one embodiment, the compositions and methods of the present invention resolve the shortcomings of current HIV therapies by achieving selective expression of quiescent HIV in the presence of anti-retroviral therapy, promoting immune cell activation, and thus depleting the reservoir of persistent HIV infection, and making it possible to not just suppress, but to eradicate HIV.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Administration” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “subject” includes any organism, preferably an animal, more preferably a mammal (e.g., rat, mouse, dog, cat, rabbit) and most preferably a human.

As used herein, the term “antagonist(s)” refers to a molecule(s) that inhibits the action of another molecule without provoking a biological response itself. In a specific embodiment, an antagonist is a molecule that binds to a receptor on a cell and blocks or dampens the biological activity of an agonist. For example, an antagonist includes an antibody or ligand that binds to a receptor on a cell and blocks or dampens binding of the native ligand to the receptor without inducing one or more signal transduction pathways. Another example of an antagonist includes an antibody or soluble receptor that competes with the native receptor on cells for binding to the native ligand, and thus, blocks or dampens one or more signal transduction pathways induced when the native receptor binds to the native ligand. Another example of an antagonist includes an antibody or soluble receptor that does not prevent the binding of the native receptor with the native ligand, but prevents signal transduction by other means (e.g., through inhibition of receptor multimerization).

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

“Anti-BTN3A antibody” refers to an antibody that specifically binds to one or more of BTN3A1, BTN3A2 and BTN3A3.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human BTN3A1, if it binds to polypeptides comprising that sequence but does not bind to proteins lacking that sequence. Due to the high homology between the BTN3A1, 3A2 or 3A3 isoforms, an antibody that specifically binds to BTN3A1, may optionally also specifically bind to BTN3A2 or BTN3A3.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Chothia” as used herein means an antibody numbering system described in Al-Lazikani et al., JMB 273:927-948 (1997).

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a BTN antibody that consists essentially of a recited amino acid sequence may also include one or more amino acids, including substitutions of one or more amino acid residues, which do not materially affect the properties of the binding compound.

“Framework region” or “FR” as used herein means the immunoglobulin variable regions excluding the CDR regions.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Butyrophilin or BTN” means human BTN1A1 (Accession #: Q13410), BTN2A1 (Accession #: Q7KYR7), BTN2A2 (Accession #: Q8WVV5), BTN3A1 (Accession #: 000481), BTN3A2 (Accession #: P78410), BTN3A3 (Accession #: 000478), BTNL2 (Accession #: Q9UIR0), human BTNL3 (Accession #: Q6UXE8), BTNL8 (Accession #: Q6UX41), human BTNL9 (Accession #: Q6UXG8), human BTNL10 (Accession #: A8MVZ5).

“BTN3A” means human CD277 and isoforms such as, human BTN3A1, BTN3A2 or BTN3A3.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

The terms “treatment regimen”, “dosing protocol” and “dosing regimen” are used interchangeably to refer to the dose and timing of administration of each therapeutic agent in a combination of the invention.

“Variable regions” or “V region” as used herein means the segment of IgG chains which is variable in sequence between different antibodies. Typically, it extends to Kabat residue 109 in the light chain and 113 in the heavy chain.

The term “treating” in its various grammatical forms in relation to the present invention refers to curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease.

The term “preventing” in the context of the present invention means that the effects of a disease state or disease causative agent has been obviated due to administration of an agent, such as those disclosed herein. A similar term in this context is “prophylaxis.”

As recited herein, “HDAC inhibitor” (e.g., SAHA) encompasses any synthetic, recombinant, or naturally-occurring histone deacetylase inhibitor, including any pharmaceutical salts or hydrates of such inhibitors, and any free acids, free bases, or other free forms of such inhibitors. “Hydroxamic acid derivative,” as used herein, refers to the class of histone deacetylase inhibitors that are hydroxamic acid derivatives. Specific examples of inhibitors are provided herein.

“Patient” or “subject” are used interchangeably herein and refer to the recipient of treatment. Mammalian and non-mammalian subjects are included. In a specific embodiment, the subject is a mammal, such as a human, canine, murine, feline, bovine, ovine, swine, or caprine. In a particular embodiment, the subject is a human. Preferably, a subject is one who has been infected with HIV, but can also encompass those who are at risk of being infected with HIV, or those who lack clinical symptoms of HIV infection, but who nevertheless may be infected with HIV present in cells in a latent form.

The terms “intermittent” or “intermittently” as used herein means stopping and starting at either regular or irregular intervals.

The term “hydrate” includes but is not limited to hemihydrate, monohydrate, dihydrate, trihydrate, and the like.

As used herein, the term “viral infection” describes a diseased state in which a virus invades healthy cells, uses the cell's reproductive machinery to multiply or replicate, release viral particles and infect other cells by the newly produced progeny viruses, with some cells dying due to viral lytic activity. Latent infection through integration of silent proviral DNA is also a possible result of viral infection.

As used herein, the term “treating viral infections” means to inhibit the replication of the particular virus, to inhibit viral transmission, and to ameliorate or alleviate the symptoms of the disease caused by the viral infection. The treatment is considered “therapeutic” if there is a reduction in viral load, decrease in mortality and/or morbidity. “Preventing viral infections” means to prevent the virus from establishing itself in the host cell.

“Latency” means a concept describing 1) the dormant state of viral activity within a population of cells, wherein viral production, viral packaging, and host cell lysis does not occur, or occurs at a very low frequency, or 2) the down-regulation or absence of viral and/or host cell gene expression within an infected cell. “Latent” in the viral context can mean that the viral genome has integrated into the host cell genome without subsequent viral expression and packaging of the viral genome into a viral capsid or other virus structure, which then causes the host cell to lyse, releasing viral particles that are free to infect other cells in the host. “Latency” in the context of the viral life cycle can also refer to a virus' “lysogenic phase.”

### Anti-BTN Antibody

In one aspect, the invention provides an antagonist anti-BTN3A antibody that activates the expression of HIV within CD4+ T cells or reactivates HIV from latency and activates CD4+ T cells. In one embodiment, the antibody increases IFNγ, IL-2 or TFNα production or T-cell proliferation in HIV latent primary CD4+ T cells and upregulates HIV transcription in CD4+ T cells. In one embodiment, the antagonist anti-BTN3A antibody cross-competes with anti-BTN3A antibody BTN20.1 or BTN103.2. In another aspect, the invention provides an antagonist anti-BTN3A antibody that activates the expression of HIV within CD4+ T cells or reactivates HIV from latency but does not activate CD4+ T cells. In one embodiment, the antibody does not increase IFNγ, IL-2 or TFNα production or T-cell proliferation in HIV latent primary CD4+ T cells but upregulates HIV transcription in CD4+ T cells. In a further aspect, the invention provides an antagonist anti-BTN3A antibody that activates CD4+ T cells but does not activate the expression of HIV within CD4+ T cells or does not reactivate HIV from latency. In one embodiment, the antibody increases IFNγ, IL-2 or TFNα production or T-cell proliferation in HIV latent primary CD4+ T cells but does not upregulate HIV transcription in CD4+ T cells.

In another embodiment, the invention provides an antibody or antigen binding fragment that specifically binds to human BTN3A1 comprising a heavy chain variable region and a light chain variable region comprising: (i) a heavy chain variable region CDR1 comprising the amino acid sequence of SEQ ID NO: 13; (ii) a heavy chain variable region CDR2 comprising the amino acid sequence of SEQ ID NO: 14; (iii) a heavy chain variable region CDR3 comprising the amino acid sequence of SEQ ID NO: 15; (iv) a light chain variable region CDR1 comprising the amino acid sequence of SEQ ID NO: 16; (v) a light chain variable region CDR2 comprising the amino acid sequence of SEQ ID NO: 17; and (vi) a light chain variable region CDR3 comprising the amino acid sequence of SEQ ID NO: 18.

In another embodiment, the invention provides an antibody or antigen binding fragment that specifically binds to human BTN3A1 comprising a heavy chain variable region and a light chain variable region comprising: (i) a heavy chain variable region CDR1 comprising the amino acid sequence of SEQ ID NO: 19; (ii) a heavy chain variable region CDR2 comprising the amino acid sequence of SEQ ID NO: 20; (iii) a heavy chain variable region CDR3 comprising the amino acid sequence of SEQ ID NO: 21; (iv) a light chain variable region CDR1 comprising the amino acid sequence of SEQ ID NO: 22; (v) a light chain variable region CDR2 comprising the amino acid sequence of SEQ ID NO: 23; and (vi) a light chain variable region CDR3 comprising the amino acid sequence of SEQ ID NO: 24.

In another embodiment, the invention provides an antibody or antigen binding fragment that specifically binds to human BTN3A1 comprising a heavy chain variable region and a light chain variable region comprising: (i) a heavy chain variable region CDR1 comprising the amino acid sequence of SEQ ID NO: 25; (ii) a heavy chain variable region CDR2 comprising the amino acid sequence of SEQ ID NO: 26; (iii) a heavy chain variable region CDR3 comprising the amino acid sequence of SEQ ID NO: 27; (iv) a light chain variable region CDR1 comprising the amino acid sequence of SEQ ID NO: 28; (v) a light chain variable region CDR2 comprising the amino acid sequence of SEQ ID NO: 29; and (vi) a light chain variable region CDR3 comprising the amino acid sequence of SEQ ID NO: 30.

In another embodiment, the invention provides an antibody or antigen binding fragment that specifically binds to human BTN3A1 comprising a heavy chain variable region and a light chain variable region comprising: (i) a heavy chain variable region CDR1 comprising the amino acid sequence of SEQ ID NO: 31; (ii) a heavy chain variable region CDR2 comprising the amino acid sequence of SEQ ID NO: 32; (iii) a heavy chain variable region CDR3 comprising the amino acid sequence of SEQ ID NO: 33; (iv) a light chain variable region CDR1 comprising the amino acid sequence of SEQ ID NO: 34; (v) a light chain variable region CDR2 comprising the amino acid sequence of SEQ ID NO: 35; and (vi) a light chain variable region CDR3 comprising the amino acid sequence of SEQ ID NO: 36.

In another embodiment, the invention provides an antibody or antigen binding fragment that specifically binds to human BTN3A1 comprising a heavy chain variable region and a light chain variable region comprising: (i) a heavy chain variable region CDR1 comprising the amino acid sequence of SEQ ID NO: 37; (ii) a heavy chain variable region CDR2 comprising the amino acid sequence of SEQ ID NO: 38; (iii) a heavy chain variable region CDR3 comprising the amino acid sequence of SEQ ID NO: 39; (iv) a light chain variable region CDR1 comprising the amino acid sequence of SEQ ID NO: 40; (v) a light chain variable region CDR2 comprising the amino acid sequence of SEQ ID NO: 41; and (vi) a light chain variable region CDR3 comprising the amino acid sequence of SEQ ID NO: 42.

In another embodiment, the invention provides an antibody or antigen binding fragment that specifically binds to human BTN3A1 comprising a heavy chain variable region and a light chain variable region comprising: (i) a heavy chain variable region CDR1 comprising the amino acid sequence of SEQ ID NO: 43; (ii) a heavy chain variable region CDR2 comprising the amino acid sequence of SEQ ID NO: 44; (iii) a heavy chain variable region CDR3 comprising the amino acid sequence of SEQ ID NO: 45; (iv) a light chain variable region CDR1 comprising the amino acid sequence of SEQ ID NO: 46; (v) a light chain variable region CDR2 comprising the amino acid sequence of SEQ ID NO: 47; and (vi) a light chain variable region CDR3 comprising the amino acid sequence of SEQ ID NO: 48.

In one aspect of the foregoing embodiments, the heavy chain framework region is of the VH1 or VH4 family. In another aspect of the foregoing embodiments, the light chain framework region is of the Vk3 or Vk1 family.

A further embodiment of the invention provides an antibody or antigen binding fragment thereof that specifically binds to human BTN3A1 comprising a heavy chain variable region comprising the amino acid sequence of SEQ ID NO:1 and a light chain variable region comprising the amino acid sequence of SEQ ID NO:2; a heavy chain variable region comprising the amino acid sequence of SEQ ID NO:3 and a light chain variable region comprising the amino acid sequence of SEQ ID NO:4; a heavy chain variable region comprising the amino acid sequence of SEQ ID NO:5 and a light chain variable region comprising the amino acid sequence of SEQ ID NO:6; a heavy chain variable region comprising the amino acid sequence of SEQ ID NO:7 and a light chain variable region comprising the amino acid sequence of SEQ ID NO:8; a heavy chain variable region comprising the amino acid sequence of SEQ ID NO:9 and a light chain variable region comprising the amino acid sequence of SEQ ID NO:10; or a heavy chain variable region comprising the amino acid sequence of SEQ ID NO:11 and a light chain variable region comprising the amino acid sequence of SEQ ID NO:12.

In one embodiment, the antibody or antigen binding fragment thereof comprises a heavy chain variable region comprising at least 90%, 95%, 96%, 97%, 98% or 99% identity to any one of SEQ ID NOs: 1, 3, 5, 7, 9, and 11; and a light chain variable region comprising at least 90%, 95%, 96%, 97%, 98% or 99% identity to any one of SEQ ID NOs: 2, 4, 6, 8, 10 and 12.

### Methods of Making Antibodies and Antigen-Binding Fragments Thereof

Methods for making an anti-BTN antibody or antigen-binding fragment thereof of the present invention comprise culturing a hybridoma cell that expresses the antibody or fragment under conditions favorable to such expression and, optionally, isolating the antibody or fragment from the hybridoma and/or the growth medium (e.g. cell culture medium).

The anti-BTN antibodies may also be produced recombinantly (e.g., in an E. coli/T7 expression system, a mammalian cell expression system or a lower eukaryote expression system). In this embodiment, nucleic acids encoding the antibody immunoglobulin molecules of the invention (e.g., V_{H} or V_{L}) may be inserted into a pET-based plasmid and expressed in the E. coli/T7 system. For example, the present invention includes methods for expressing an antibody or antigen-binding fragment thereof or immunoglobulin chain thereof in a host cell (e.g., bacterial host cell such as E. coli such as BL21 or BL21DE3) comprising expressing T7 RNA polymerase in the cell which also includes a polynucleotide encoding an immunoglobulin chain that is operably linked to a T7 promoter. For example, in an embodiment of the invention, a bacterial host cell, such as a E. coli, includes a polynucleotide encoding the T7 RNA polymerase gene operably linked to a lac promoter and expression of the polymerase and the immunoglobulin chain is induced by incubation of the host cell with IPTG (isopropyl-beta-D-thiogalactopyranoside).

There are several methods used to produce recombinant antibodies that are known in the art. One example of a method for recombinant production of antibodies is disclosed in U.S. Pat. No. 4,816,567.

Transformation by any known method can be used to introduce polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455.

Eukaryotic and prokaryotic host cells, including mammalian cells, can be used as hosts for expression of the antibodies or fragments or immunoglobulin chains and are well known in the art, including many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. Fungal cells include yeast and filamentous fungus cells including, for example, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa, Pichia sp., any Saccharomyces sp., Hansenula polymorpha, any Kluyveromyces sp., Candida albicans, any Aspergillus sp., Trichoderma reesei, Chrysosporium lucknowense, any Fusarium sp., Yarrowia lipolytica, and Neurospora crassa. When recombinant expression vectors encoding the heavy chain or antigen-binding portion or fragment thereof and the light chain and/or antigen-binding fragment thereof are introduced into mammalian host cells, the antibodies are produced by culturing the host cells for a period of time sufficient to allow for expression of the antibody or fragment or chain in the host cells or secretion of the antibody or fragment into the culture medium in which the host cells are grown.

Antibodies and antigen-binding fragments thereof and immunoglobulin chains can be recovered from the culture medium using standard protein purification methods. Further, expression of antibodies and antigen-binding fragments thereof and immunoglobulin chains of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4. Thus, in an embodiment of the invention, the mammalian host cells (e.g., CHO) lack a glutamine synthetase gene and are grown in the absence of glutamine in the medium wherein however, the polynucleotide encoding the immunoglobulin chain comprises a glutamine synthetase gene which complements the lack of the gene in the host cell.

The present invention further includes anti-BTN antigen-binding fragments of the anti-BTN antibodies. The antibody fragments include F(ab)₂ fragments, which may be produced by enzymatic cleavage of an IgG by, for example, pepsin. Fab fragments may be produced by, for example, reduction of F(ab)₂ with dithiothreitol or mercaptoethylamine.

Immunoglobulins may be assigned to different classes depending on the amino acid sequences of the constant domain of their heavy chains. There are at least five major classes of immunoglobulins: IgA, IgD, IgE, IgG and IgM, and several of these may be further divided into subclasses (isotypes), e.g. IgG1, IgG2, IgG3 and IgG4; IgA1 and IgA2. The invention comprises antibodies and antigen-binding fragments of any of these classes or subclasses of antibodies.

In one embodiment, the antibody or antigen-binding fragment comprises a heavy chain constant region, e.g. a human constant region, such as γ1, γ2, γ3, or γ4 human heavy chain constant region or a variant thereof. In another embodiment, the antibody or antigen-binding fragment comprises a light chain constant region, e.g. a human light chain constant region, such as lambda or kappa human light chain region or variant thereof. By way of example, and not limitation the human heavy chain constant region can be γ4 and the human light chain constant region can be kappa. In an alternative embodiment, the Fc region of the antibody is γ4 with a Ser228Pro mutation (Schuurman, J et. al., Mol. Immunol. 38: 1-8, 2001).

In one embodiment, the antibody or antigen-binding fragment comprises a heavy chain constant region of the IgG1 subtype. In one embodiment, the antibody or antigen-binding fragment comprises a heavy chain constant region of the IgG1 subtype with an N297A mutation. In one embodiment, the antibody or antigen-binding fragment comprises a heavy chain constant region of the IgG2 subtype. In one embodiment, the antibody or antigen-binding fragment comprises a heavy chain constant region of the IgG4 subtype.

### Antibody Engineering

Further included are embodiments in which the anti-BTN antibodies and antigen-binding fragments thereof are engineered antibodies to include modifications to framework residues within the variable domains of a parental mouse monoclonal antibody, e.g. to improve the properties of the antibody or fragment. Typically, such framework modifications are made to decrease the immunogenicity of the antibody or fragment. This is usually accomplished by replacing non-CDR residues in the variable domains (i.e. framework residues) in a parental (e.g. rodent) antibody or fragment with analogous residues from the immune repertoire of the species in which the antibody is to be used, e.g. human residues in the case of human therapeutics. Such an antibody or fragment is referred to as a “humanized” antibody or fragment. In some cases it is desirable to increase the affinity, or alter the specificity of an engineered (e.g. humanized) antibody. One approach is to “back-mutate” one or more framework residues to the corresponding germline sequence. More specifically, an antibody or fragment that has undergone somatic mutation can contain framework residues that differ from the germline sequence from which the antibody is derived. Such residues can be identified by comparing the antibody or fragment framework sequences to the germline sequences from which the antibody or fragment is derived. Another approach is to revert to the original parental (e.g., rodent) residue at one or more positions of the engineered (e.g. humanized) antibody, e.g. to restore binding affinity that may have been lost in the process of replacing the framework residues. (See, e.g., U.S. Pat. Nos. 5,693,762, 5,585,089 and 5,530,101.)

In certain embodiments, the anti-BTN antibodies and antigen-binding fragments thereof are engineered (e.g. humanized) to include modifications in the framework and/or CDRs to improve their properties. Such engineered changes can be based on molecular modeling. A molecular model for the variable region for the parental (non-human) antibody sequence can be constructed to understand the structural features of the antibody and used to identify potential regions on the antibody that can interact with the antigen. Conventional CDRs are based on alignment of immunoglobulin sequences and identifying variable regions. Kabat et al., (1991) Sequences of Proteins of Immunological Interest, Kabat, et al.; National Institutes of Health, Bethesda, Md.; 5^{th} ed.; NIH Publ. No. 91-3242; Kabat (1978) Adv. Prot. Chem. 32:1-75; Kabat, et al., (1977) J. Biol. Chem. 252:6609-6616. Chothia and coworkers carefully examined conformations of the loops in crystal structures of antibodies and proposed hypervariable loops. Chothia, et al., (1987) J Mol. Biol. 196:901-917 or Chothia, et al., (1989) Nature 342:878-883. There are variations between regions classified as “CDRs” and “hypervariable loops”. Later studies (Raghunathan et al, (2012) J. Mol Recog. 25, 3, 103-113) analyzed several antibody-antigen crystal complexes and observed that the antigen binding regions in antibodies do not necessarily conform strictly to the “CDR” residues or “hypervariable” loops. The molecular model for the variable region of the non-human antibody can be used to guide the selection of regions that can potentially bind to the antigen. In practice, the potential antigen binding regions based on model differ from the conventional “CDR”s or “hyper variable” loops. Commercial scientific software such as MOE (Chemical Computing Group) can be used for molecular modeling. Human frameworks can be selected based on best matches with the non-human sequence both in the frameworks and in the CDRs. For FR4 (framework 4) in VH, VJ regions for the human germlines are compared with the corresponding non-human region. In the case of FR4 (framework 4) in VL, J-kappa and J-Lambda regions of human germline sequences are compared with the corresponding non-human region. Once suitable human frameworks are identified, the CDRs are grafted into the selected human frameworks. In some cases certain residues in the VL-VH interface can be retained as in the non-human (parental) sequence. Molecular models can also be used for identifying residues that can potentially alter the CDR conformations and hence binding to antigen. In some cases, these residues are retained as in the non-human (parental) sequence. Molecular models can also be used to identify solvent exposed amino acids that can result in unwanted effects such as glycosylation, deamidation and oxidation. Developability filters can be introduced early on in the design stage to eliminate/minimize these potential problems.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Pat. No. 7,125,689.

### Altered Effector Function

In some embodiments, the Fc region of an anti-BTN antibody is modified to increase the ability of the antibody or antigen-binding fragment to mediate effector function and/or to increase their binding to the Fcgamma receptors (FcγRs).

The term “Effector Function” as used herein is meant to refer to one or more of Antibody Dependant Cell mediated Cytotoxic activity (ADCC), Complement-dependant cytotoxic activity (CDC) mediated responses, Fc-mediated phagocytosis or antibody dependant cellular phagocytosis (ADCP) and antibody recycling via the FcRn receptor.

The interaction between the constant region of an antigen binding protein and various Fc receptors (FcR) including FcgammaRI (CD64), FcgammaRII (CD32) and FcgammaRIII (CD16) is believed to mediate the effector functions, such as ADCC and CDC, of the antigen binding protein.

Effector function can be measured in a number of ways including for example via binding of the FcgammaRIII to Natural Killer cells or via FcgammaRI to monocytes/macrophages to measure for ADCC effector function. For example, an antigen binding protein of the present invention can be assessed for ADCC effector function in a Natural Killer cell assay. Examples of such assays can be found in Shields et al, 2001 J. Biol. Chem., Vol. 276, p 6591-6604; Chappel et al, 1993 J. Biol. Chem., Vol 268, p 25124-25131; Lazar et al, 2006 PNAS, 103; 4005-4010.

Human IgG1 constant regions containing specific mutations or altered glycosylation on residue Asn297 have been shown to reduce binding to Fc receptors. In other cases, mutations have also been shown to enhance ADCC and CDC (Lazar et al. PNAS 2006, 103; 4005-4010; Shields et al. J Biol Chem 2001, 276; 6591-6604; Nechansky et al. Mol Immunol, 2007, 44; 1815-1817).

In one embodiment of the present invention, such mutations are in one or more of positions selected from 239, 332 and 330 (IgG1), or the equivalent positions in other IgG isotypes. Examples of suitable mutations are S239D and I332E and A330L. In one embodiment, the antigen binding protein is mutated at positions 239 and 332, for example S239D and I332E, or in a further embodiment it is mutated at three or more positions selected from 239 and 332 and 330, for example S239D and I332E and A330L. (EU index numbering).

In an alternative embodiment of the present invention, there is provided an antibody comprising a heavy chain constant region with an altered glycosylation profile such that the antigen binding protein has enhanced effector function. For example, the antibody has enhanced ADCC or enhanced CDC, or it has both enhanced ADCC and CDC effector function. Examples of suitable methodologies to produce antigen binding proteins with an altered glycosylation profile are described in WO2003011878, WO2006014679 and EP1229125.

### Histone Deacetylases and Histone Deacetylase Inhibitors

Histone deacetylases (HDACs) include enzymes that catalyze the removal of acetyl groups from lysine residues in the amino terminal tails of the nucleosomal core histones. As such, HDACs, together with histone acetyl transferases (HATs) regulate the acetylation status of histones. Histone acetylation affects gene expression and inhibitors of HDACs, such as the hydroxamic acid-based hybrid polar compound suberoylanilide hydroxamic acid (SAHA) induce growth arrest, differentiation, and/or apoptosis of transformed cells in vitro and inhibit tumor growth in vivo.

HDACs can be divided into three classes based on structural homology. Class I HDACs (HDACs 1, 2, 3, and 8) bear similarity to the yeast RPD3 protein, are located in the nucleus and are found in complexes associated with transcriptional co-repressors. Class II HDACs (HDACs 4, 5, 6, 7 and 9) are similar to the yeast HDA1 protein and have both nuclear and cytoplasmic subcellular localization. Class III HDACs form a structurally distant class of NAD dependent enzymes that are related to the yeast SIR2 proteins and are not inhibited by hydroxamic acid-based HDAC inhibitors.

Histone deacetylase inhibitors or HDAC inhibitors are compounds that are capable of inhibiting the deacetylation of histones in vivo, in vitro or both. As such, HDAC inhibitors inhibit the activity of at least one histone deacetylase. As a result of inhibiting the deacetylation of at least one histone, an increase in acetylated histone occurs and accumulation of acetylated histone is a suitable biological marker for assessing the activity of HDAC inhibitors. Therefore, procedures that can assay for the accumulation of acetylated histones can be used to determine the HDAC inhibitory activity of compounds of interest. It is understood that compounds that can inhibit histone deacetylase activity can also bind to other substrates and as such can inhibit other biologically active molecules such as enzymes. It is also understood that the compounds of the present invention are capable of inhibiting any of the histone deacetylases set forth above, or any other histone deacetylases.

For example, in patients receiving HDAC inhibitors, the accumulation of acetylated histones in peripheral mononuclear cells as well as in tissue treated with HDAC inhibitors can be determined against a suitable control.

HDAC inhibitory activity of a particular compound can be determined in vitro using, for example, an enzymatic assay which shows inhibition of at least one histone deacetylase. Further, determination of the accumulation of acetylated histones in cells treated with a particular composition can be determinative of the HDAC inhibitory activity of a compound.

Assays for the accumulation of acetylated histones are well known in the literature. See, for example, Marks, P. A. et al., J. Natl. Cancer Inst., 92: 1210-1215 (2000); Butler, L. M. et al., Cancer Res. 60: 5165-5170 (2000); Richon, V. M. et al., Proc. Natl. Acad. Sci., USA, 95: 3003-3007 (1998); and Yoshida, M. et al., J. Biol. Chem., 265: 17174-17179 (1990).

For example, an enzymatic assay to determine the activity of an HDAC inhibitor compound can be conducted as follows. Briefly, the effect of an HDAC inhibitor compound on affinity purified human epitope-tagged (Flag) HDAC1 can be assayed by incubating the enzyme preparation in the absence of substrate on ice for about 20 minutes with the indicated amount of inhibitor compound. Substrate ([³H]acetyl-labeled murine erythroleukemia cell-derived histone) can be added and the sample can be incubated for 20 minutes at 37° C. in a total volume of 30 μL. The reaction can then be stopped and released acetate can be extracted and the amount of radioactivity release determined by scintillation counting. An alternative assay useful for determining the activity of an HDAC inhibitor compound is the “HDAC Fluorescent Activity Assay; Drug Discovery Kit-AK-500” available from BIOMOL® Research Laboratories, Inc., Plymouth Meeting, Pa.

In vivo studies can be conducted as follows. Animals, for example, mice, can be injected intraperitoneally with an HDAC inhibitor compound. Selected tissues, for example, brain, spleen, liver etc, can be isolated at predetermined times, post administration. Histones can be isolated from tissues essentially as described by Yoshida et al., J. Biol. Chem. 265: 17174-17179 (1990). Equal amounts of histones (about 1 μg) can be electrophoresed on 15% SDS-polyacrylamide gels and can be transferred to Hybond-P filters (available from Amersham). Filters can be blocked with 3% milk and can be probed with a rabbit purified polyclonal anti-acetylated histone H4 antibody (αAc-H4) and anti-acetylated histone H3 antibody (αAc-H3) (Upstate Biotechnology, Inc.). Levels of acetylated histone can be visualized using a horseradish peroxidase-conjugated goat anti-rabbit antibody (1:5000) and the SuperSignal chemiluminescent substrate (Pierce). As a loading control for the histone protein, parallel gels can be run and stained with Coomassie Blue (CB).

Non-limiting examples of such HDAC inhibitors are set forth below. It is understood that the present invention includes any salts, crystal structures, amorphous structures, hydrates, derivatives, metabolites, stereoisomers, structural isomers, and prodrugs of the HDAC inhibitors described herein.

A. Hydroxamic Acid Derivatives such as Suberoylanilide hydroxamic acid (SAHA) (Richon et al., Proc. Natl. Acad. Sci. USA 95, 3003-3007 (1998)); m-Carboxycinnamic acid bishydroxamide (CBHA) (Richon et al., supra); Pyroxamide; Trichostatin analogues such as Trichostatin A (TSA) and Trichostatin C (Koghe et al. Biochem. Pharmacol. 56: 1359-1364 (1998)); Salicylbishydroxamic acid (Andrews et al., International J. Parasitology 30, 761-768 (2000)); Suberoyl bishydroxamic acid (SBHA) (U.S. Pat. No. 5,608,108); Azelaic bishydroxamic acid (ABHA) (Andrews et al., supra); Azelaic-1-hydroxamate-9-anilide (AAHA) (Qiu et al., Mol. Biol. Cell 11, 2069-2083 (2000)); 6-(3-Chlorophenylureido) carpoic hydroxamic acid (3Cl-UCHA); Oxamflatin [(2E)-5-[3-[(phenylsufonyl) aminol phenyl]-pent-2-en-4-ynohydroxamic acid] (Kim et al. Oncogene, 18: 2461 2470 (1999)); A-161906, Scriptaid (Su et al. Cancer Research, 60: 3137-3142 (2000)); PXD-101 (Prolifix); LAQ-824; CHAP; MW2796 (Andrews et al., supra); MW2996 (Andrews et al., supra); or any of the hydroxamic acids disclosed in U.S. Pat. Nos. 5,369,108, 5,932,616, 5,700,811, 6,087,367, and 6,511,990.

B. Cyclic Tetrapeptides such as Trapoxin A (TPX)-cyclic tetrapeptide (cyclo-(L-phenylalanyl-L-phenylalanyl-D-pipecolinyl-L-2-amino-8-oxo-9,10-epoxy decanoyl)) (Kijima et al., J. Biol. Chem. 268, 22429-22435 (1993)); FR901228 (FK 228, depsipeptide) (Nakajima et al., Ex. Cell Res. 241, 126-133 (1998)); FR225497 cyclic tetrapeptide (H. Mori et al., PCT Application WO 00/08048 (17 Feb. 2000)); Apicidin cyclic tetrapeptide [cyclo(N—O-methyl-L-tryptophanyl-L-isoleucinyl-D-pipecolinyl-L-2-amino-8-oxodecanoyl)](Darkin-Rattray et al., Proc. Natl. Acad. Sci. USA 93, 13143-13147 (1996)); Apicidin Ia, Apicidin Ib, Apicidin Ic, Apicidin IIa, and Apicidin IIb (P. Dulski et al., PCT Application WO 97/11366); CHAP, HC-toxin cyclic tetrapeptide (Bosch et al., Plant Cell 7, 1941-1950 (1995)); WF27082 cyclic tetrapeptide (PCT Application WO 98/48825); and Chlamydocin (Bosch et al., supra).

C. Short chain fatty acid (SCFA) derivatives such as: Sodium Butyrate (Cousens et al., J. Biol. Chem. 254, 1716-1723 (1979)); Isovalerate (McBain et al., Biochem. Pharm. 53: 1357-1368 (1997)); Valerate (McBain et al., supra); 4-Phenylbutyrate (4-PBA) (Lea and Tulsyan, Anticancer Research, 15, 879-873 (1995)); Phenylbutyrate (PB) (Wang et al., Cancer Research, 59, 2766-2799 (1999)); Propionate (McBain et al., supra); Butyramide (Lea and Tulsyan, supra); Isobutyramide (Lea and Tulsyan, supra); Phenylacetate (Lea and Tulsyan, supra); 3-Bromopropionate (Lea and Tulsyan, supra); Tributyrin (Guan et al., Cancer Research, 60, 749-755 (2000)); Valproic acid, Valproate, and Pivanex™.

D. Benzamide derivatives such as CI-994; MS-275 [N-(2-aminophenyl)-4-[N-(pyridin-3-yl methoxycarbonyl) aminomethyl] benzamide] (Saito et al., Proc. Natl. Acad. Sci. USA 96, 4592-4597 (1999)); and 3′-amino derivative of MS-275 (Saito et al., supra).

E. Electrophilic ketone derivatives such as Trifluoromethyl ketones (Frey et al, Bioorganic & Med. Chem. Lett., 12, 3443-3447 (2002); U.S. Pat. No. 6,511,990) and α-keto amides such as N-methyl-α-ketoamides.

F. Other HDAC Inhibitors such as natural products, psammaplins, and Depudecin (Kwon et al. PNAS 95: 3356-3361 (1998)).

HDAC inhibitors include those disclosed in U.S. Pat. Nos. 5,369,108, 5,932,616, 5,700,811, 6,087,367, and 6,511,990, issued to some of the present inventors, the entire contents of which are incorporated herein by reference, non-limiting examples of which are set forth below:

Specific HDAC inhibitors include suberoylanilide hydroxamic acid (SAHA; N-Hydroxy-N-phenyl octanediamide), which is represented by the following structural formula:

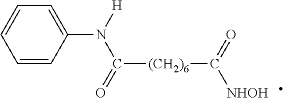

Other examples of such compounds and other HDAC inhibitors can be found in U.S. Pat. No. 5,369,108, issued on Nov. 29, 1994, U.S. Pat. No. 5,700,811, issued on Dec. 23, 1997, U.S. Pat. No. 5,773,474, issued on Jun. 30, 1998, U.S. Pat. No. 5,932,616, issued on Aug. 3, 1999 and U.S. Pat. No. 6,511,990, issued Jan. 28, 2003, all to Breslow et al.; U.S. Pat. No. 5,055,608, issued on Oct. 8, 1991, U.S. Pat. No. 5,175,191, issued on Dec. 29, 1992 and U.S. Pat. No. 5,608,108, issued on Mar. 4, 1997, all to Marks et al.; as well as Yoshida, M., et al., Bioassays 17, 423-430 (1995); Saito, A., et al., PNAS USA 96, 4592-4597, (1999); Furamai R. et al., PNAS USA 98 (1), 87-92 (2001); Komatsu, Y., et al., Cancer Res. 61(11), 4459-4466 (2001); Su, G. H., et al., Cancer Res. 60, 3137-3142 (2000); Lee, B. I. et al., Cancer Res. 61(3), 931-934; Suzuki, T., et al., J. Med. Chem. 42(15), 3001-3003 (1999); published PCT Application WO 01/18171 published on Mar. 15, 2001 to Sloan-Kettering Institute for Cancer Research and The Trustees of Columbia University; published PCT Application WO 02/246144 to Hoffmann-La Roche; published PCT Application WO 02/22577 to Novartis; published PCT Application WO 02/30879 to Prolifix; published PCT Applications WO 01/38322 (published May 31, 2001), WO 01/70675 (published on Sep. 27, 2001) and WO 00/71703 (published on Nov. 30, 2000) all to Methylgene, Inc.; published PCT Application WO 00/21979 published on Oct. 8, 1999 to Fujisawa Pharmaceutical Co., Ltd.; published PCT Application WO 98/40080 published on Mar. 11, 1998 to Beacon Laboratories, L.L.C.; Curtin M. (Current patent status of HDAC inhibitors Expert Opin. Ther. Patents 12(9): 1375-1384 (2002) and references cited therein); and U.S. patent application Ser. No. 10/600,132 (Publication No. 20040122101, filed Jun. 19, 2003) and Ser. No. 11/981,367 (filed Oct. 30, 2007).

SAHA or any of the other HDACs can be synthesized according to the method set forth in U.S. Pat. Nos. 5,369,108, 5,700,811, 5,932,616 and 6,511,990, the contents of which are incorporated by reference in their entirety, or according to any other method known to a person skilled in the art.

Specific non-limiting examples of HDAC inhibitors are provided in the Table 1 below. It should be noted that the present invention encompasses any compounds which are structurally similar to the compounds represented below, and which are capable of inhibiting histone deacetylases.

TABLE 1

| Name | Structure |
|---|---|
| MS-275 | 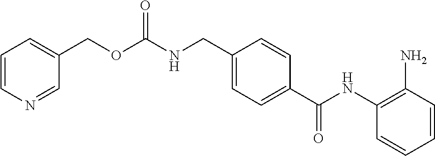 [SMILES: Nc1ccccc1NC(=O)c1ccc(CNC(=O)OCc2cccnc2)cc1] |
| DEPSIPEPTIDE | 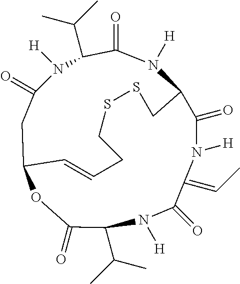 [SMILES: C/C=C1\NC(=O)[C@H]2CSSCC/C=C/[C@H](CC(=O)N[C@H](C(C)C)C(=O)N2)OC(=O)[C@H](C(C)C)NC1=O] |
| CI-994 | 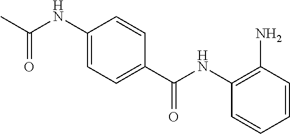 [SMILES: CC(=O)Nc1ccc(C(=O)Nc2ccccc2N)cc1] |
| Apicidin | 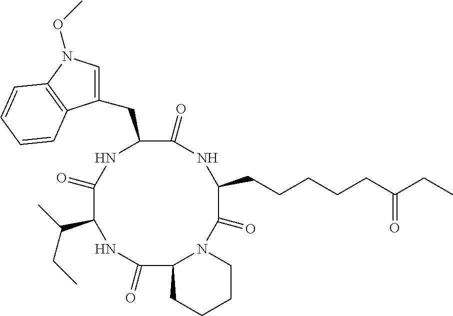 [SMILES: CCC(=O)CCCCC[C@@H]1NC(=O)[C@H](Cc2cn(OC)c3ccccc23)NC(=O)[C@H](C(C)CC)NC(=O)[C@@H]2CCCCN2C1=O] |
| A-161906 | 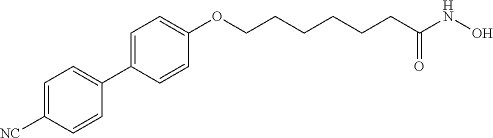 [SMILES: N#Cc1ccc(-c2ccc(OCCCCCCC(=O)NO)cc2)cc1] |
| Scriptaid | 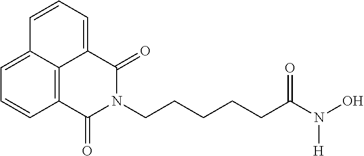 [SMILES: O=C(CCCCCN1C(=O)c2cccc3cccc(c23)C1=O)NO] |
| PXD-101 | 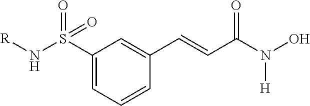 |
| CHAP | 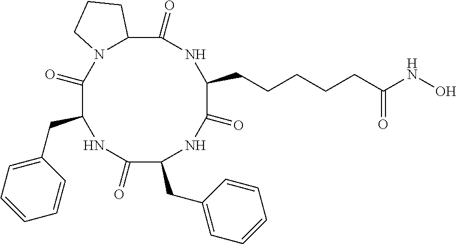 [SMILES: O=C(CCCCC[C@@H]1NC(=O)C2CCCN2C(=O)[C@H](Cc2ccccc2)NC(=O)[C@H](Cc2ccccc2)NC1=O)NO] |
| LAQ-824 | 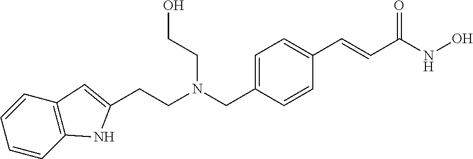 [SMILES: O=C(/C=C/c1ccc(CN(CCO)CCc2cc3ccccc3[nH]2)cc1)NO] |
| Butyric Acid | 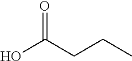 |
| Depudecin | 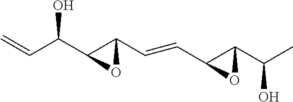 |
| Oxamflatin | 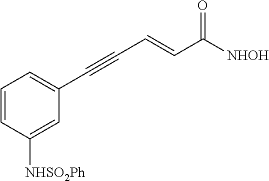 |
| Trichostatin C | 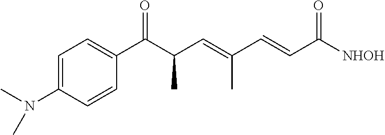 |

### Administration of Anti-Viral Agents or Latency Reversing Agents

In this invention, the BTN antibody can be used in combination with one or more anti-retroviral agents, including: (1) nucleoside reverse transcriptase inhibitors, (2) non-nucleoside reverse transcriptase inhibitors, (3) protease inhibitors, (4) virus uptake/adsorption inhibitors, (5) virus receptor antagonists, (6) viral fusion inhibitors, (7) viral integrase inhibitors, (8) transcription inhibitors (9) entry inhibitor, or (10) other anti-retroviral agents used in treatment of HIV infection.

TABLE 2
Antiviral Agents for Treating HIV infection or AIDS

| Name | Type |
|---|---|
| abacavir, abacavir sulfate, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, atazanavir sulfate, Reyataz ® | PI |
| AZT, zidovudine, azidothymidine, Retrovir ® | nRTI |
| Capravirine | nnRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, delavirdine mesylate, DLV, Rescriptor ® | nnRTI |
| dolutegravir, Tivicay ® | InI |
| doravirine, MK-1439 | nnRTI |
| efavirenz, EFV, Sustiva ®, Stocrin ® | nnRTI |
| EFdA (4′-ethynyl-2-fluoro-2′-deoxyadenosine) | nRTI |
| Elvitegravir | InI |
| emtricitabine, FTC, Emtriva ® | nRTI |
| emivirine, Coactinon ® | nnRTI |
| enfuvirtide, Fuzeon ® | FI |
| enteric coated didanosine, Videx EC ® | nRTI |
| etravirine, TMC-125 | nnRTI |
| fosamprenavir calcium, Lexiva ® | PI |
| indinavir, indinavir sulfate, Crixivan ® | PI |
| lamivudine, 3TC, Epivir ® | nRTI |
| lamivudine + zidovudine, Combivir ® | nRTI |
| Lopinavir | PI |
| lopinavir + ritonavir, Kaletra ® | PI |
| maraviroc, Selzentry ® | EI |
| nelfinavir, nelfinavir mesylate, Viracept ® | PI |
| nevirapine, NVP, Viramune ® | nnRTI |
| PPL-100 (also known as PL-462) (Ambrilia) | PI |
| raltegravir, MK-0518, Isentress ™ | InI |
| Rilpivirine | nnRTI |
| ritonavir, Norvir ® | PI |
| saquinavir, saquinavir mesylate, Invirase ®, Fortovase ® | PI |
| stavudine, d4T, didehydrodeoxythymidine, Zerit ® | nRTI |
| tipranavir, Aptivus ® | PI |
| vicriviroc, vicriviroc maleate | EI |
| Tenofovir disoproxil fumarate | nRTI |
| Tenofovir alafenamide fumarate | nRTI |

EI = entry inhibitor; FI = fusion inhibitor; InI = integrase inhibitor; PI = protease inhibitor; nRTI = nucleoside reverse transcriptase inhibitor; nnRTI = non-nucleoside reverse transcriptase inhibitor. Some of the drugs listed in the table are used in a salt form; e.g., abacavir sulfate, delavirdine mesylate, indinavir sulfate, atazanavir sulfate, nelfinavir mesylate, saquinavir mesylate.

The BTN antibody can also be used in combination with one or more latency reversing agents, such as epigenetic modulators, molecules that alter cell metabolism and transcription, or cell signaling molecules. Latency reversing agents include but are not limited to epigenetic modulators (HDAC inhibitor, Bromodomain inhibitors, histone methyl transferase inhibitors), NFkB activators, NFAT activators, PTEFb activators, Toll-like receptor agonists, protein kinase C agonists, cytokines (IL-15 etc.), antibodies to immunomodulator receptors. In one embodiment, the histone deactylase inhibitor is SAHA or a pharmaceutically acceptable salt or hydrate thereof.

The anti-viral or latency reversing agents may be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. These agents can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions comprising anti-viral agents of the present invention and of ingredients suitable for use in said compositions is provided in Remington's Pharmaceutical Sciences, 18^{th} edition, edited by A. R. Gennaro, Mack Publishing Co., 1990 and in Remington—The Science and Practice of Pharmaev, 21^{st} Edition, Lippincott Williams & Wilkins, 2005.

The anti-viral or latency reversing agents can be administered orally in a dosage range of about 0.001 to about 1000 mg/kg of mammal (e.g., human) body weight per day in a single dose or in divided doses. One preferred dosage range is about 0.01 to about 500 mg/kg body weight per day orally in a single dose or in divided doses. Another preferred dosage range is about 0.1 to about 100 mg/kg body weight per day orally in single or divided doses. For oral administration, the compositions can be provided in the form of tablets or capsules containing about 1.0 to about 500 milligrams of the active ingredient. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. The anti-viral agents and latency reversing agents can also be administered according to known procedures, known dosages, and known dosing regimens. Further description of routes of administration, dosages, and dosage forms for anti-retroviral agents and latency reversing agents is provided in The Merck Manual of Therapy and Diagnosis, 18^{th} Edition, John Wiley & Sons, New York, N.Y.

### Administration of Anti-BTN Antibodies

Selecting a dosage regimen (also referred to herein as an administration regimen) for a combination therapy of the invention depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of each therapeutic agent delivered to the patient consistent with an acceptable level of side effects. Accordingly, the dose amount and dosing frequency of each biotherapeutic and chemotherapeutic agent in the combination depends in part on the particular therapeutic agent, the severity of the disease being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies. Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the disease to be treated and biomarkers of response to one or more of the therapeutic agents in the combination therapy.

Biotherapeutic agents in a combination therapy of the invention may be administered by continuous infusion, or by doses at intervals of, e.g., daily, every other day, three times per week, or one time each week, two weeks, three weeks, monthly, bimonthly, etc. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See. e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144.

Pharmaceutically acceptable excipients of the present disclosure include for instance, solvents, bulking agents, buffering agents, tonicity adjusting agents, and preservatives (see, e.g., Pramanick et al., Pharma Times, 45:65-77, 2013). In some embodiments the pharmaceutical compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent). The pharmaceutical compositions of the present disclosure are suitable for parenteral administration.

In some embodiments, the pharmaceutical compositions comprise an aqueous vehicle as a solvent. Suitable vehicles include for instance sterile water, saline solution, phosphate buffered saline, and Ringer's solution. In some embodiments, the composition is isotonic.

The pharmaceutical compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 4 to 9. In some embodiments, the pH is greater than (lower limit) 4, 5, 6, 7 or 8. In some embodiments, the pH is less than (upper limit) 9, 8, 7, 6 or 5. That is, the pH is in the range of from about 4 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical compositions may comprise a preservative. Suitable preservatives include for instance antioxidants and antimicrobial agents. However, in preferred embodiments, the pharmaceutical composition is prepared under sterile conditions and is in a single use container, and thus does not necessitate inclusion of a preservative.

### Combination Administration

In various aspects of the invention, the treatment procedures are performed sequentially in any order, concurrently, consequently, alternatively or a combination thereof. For example, the first treatment procedure, e.g., administration of the BTN antibody, can take place prior to the second treatment procedure, e.g., the anti-viral agent or latency reversing agent, after the second treatment with the anti-viral agent or latency reversing agent, at the same time as the second treatment with the anti-viral agent or latency reversing agent, or a combination thereof.

### EXAMPLES

The examples are presented in order to more fully illustrate the various embodiments of the invention. These examples should in no way be construed as limiting the scope of the invention recited in the appended claims.

An Aptamer screen revealed association of butyrophilins with HIV infected αβ CD4+ T cells (below). Immuno-pulldown of CD4+ T cells from HIV+ ART suppressed donors using an anti-BTN3A antibody showed enrichment of HIV-1 infected cells in some donors. BTNs also suppressed anti-CD3 antibody-induced activation of αβ CD4+ T cells in vitro, and functional blockade of BTN resulted in increased viral transcription upon anti-CD3 antibody stimulation. This data implicates BTN3A as a target for HIV transcriptional regulation.

### Example 1: Identification of Novel Biomarkers/Targets of HIV Latency Using Aptamer Screens

### A. CD4+ T Cell Model of HIV Latency/Production of Latent HIV-1-Infected Primary Cells:

Latent HIV-1 infected primary CD4+ T cells were generated following a protocol licensed from the laboratory of Jonathan Karn (Case Western Reserve University, Cleveland, Ohio) [2]. Briefly, naive CD4⁺ T cells isolated from 2 healthy human donors were propagated for 6 days in growth medium supplemented with Dynabeads Human T-Activator CD3/CD28 (25 μl/10⁶ cells) and Th17 polarizing cytokines: TGF-β1 (5 ng/ml), IL-4 (10 ng/ml), IFNγ (10 ng/ml), IL-1β (10 ng/ml), IL-6 (30 ng/ml), IL-23 (50 ng/ml), IL-8 (15 ng/ml), IL-10 (10 ng/ml). Th17 polarized cells were subsequently infected with a VSV-G-pseudotyped HIV-1 Nef⁺ virus that expresses mouse CD8a and d2EGFP. HIV-1-infected cells were then purified with a mouse anti-CD8a selection kit and quiescence was induced in the infected cells by culturing in growth media containing low concentrations of IL-2 (15 IU/ml) and IL-23 (12.5 μg/ml) for 2 weeks. Reactivation of HIV-1 proviruses from latently infected cells was performed by treatment with Dynabeads Human T-Activator CD3/CD28 (25 μl/10⁶ cells) or PMA/Ionomycin.

### B. Aptamer Screens on Latent HIV-1-Infected Primary Cells and Protein Target ID:

i. Aptamer Screens:

Caris Life Sciences (Phoenix, Ariz.) conducted aptamer screens on HIV-1 infected and uninfected CD4+ T cells prepared from 2 donors (as described in section 1A). Caris aptamer screening technology is described in WO2018064229.

Briefly, aptamer screens were conducted on uninfected, active HIV-1 infected and latent HIV-1 infected cell populations using the ADAPTamer™ library (Caris Life Sciences). Approximately, 500,000 CD4+ T cells with latent HIV were used for positive selection and a mixture of 500,000 uninfected CD4+ T cells and 500,000 cells with active HIV (same donor) were used for negative selection. After six rounds of enrichment, library complexities were approximately 5×10⁷ and probing was performed under conditions similar to those used for enrichment. Protein target ID and affinity-capture by aptamers was carried out, followed by LC-MS/MS and data analyses.

The aptamer screen identified butyrophilin-like protein 8 (BTNL8) as a potential protein target that was enriched on latent HIV-1 infected cells as compared to uninfected and active HIV-1 infected cells in one of the two donors tested. Confirmation of ADAPT protein hits is required to determine if BTNL8, and other members of the butyrophilin family, are valid protein targets.

### C. Confirmation of ADAPT Protein Hits by Flow Cytometry:

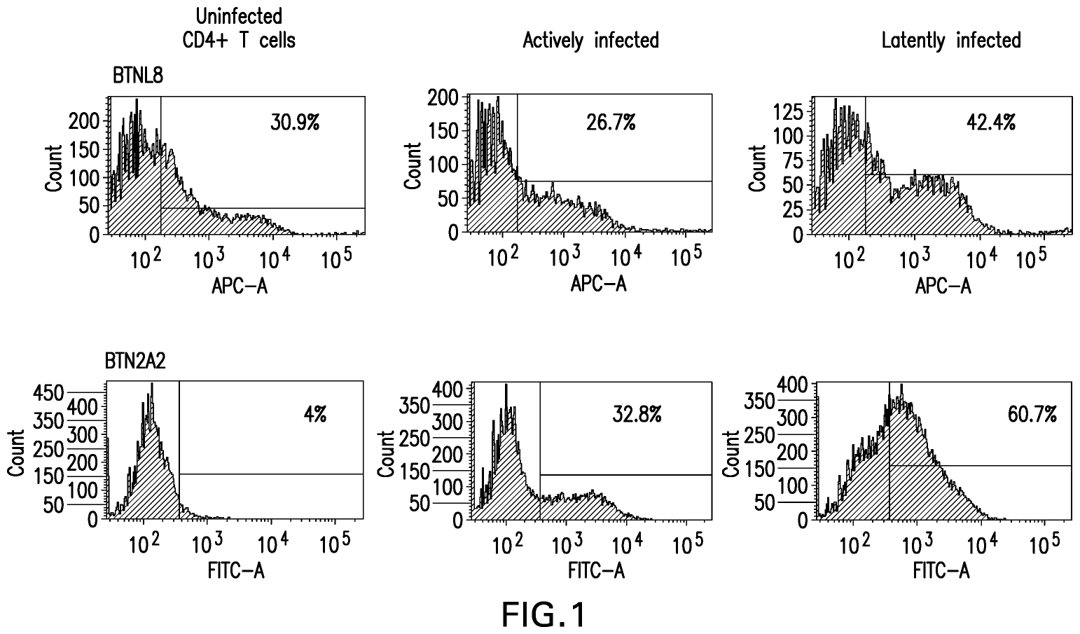

Confirmation of butyrophilin protein hits identified from ADAPT profiling was performed using flow cytometry with antibodies procured from commercial sources. Briefly, HIV infected (active and latent infection) or uninfected primary CD4+ T cells prepared from healthy donors were assessed by flow cytometry for butyrophilin protein expression using anti-BTNL8 (clone BTN9.2, Thermo Fisher) and anti-BTN2A2 antibodies (polyclonal, Novus Biologicals or R&D Systems) (FIG. 1).

Higher levels of BTN2A2 and BTN8 were expressed on HIV infected primary CD4+ T cells as compared to uninfected cells (FIG. 1). Expression levels were elevated on latently infected cells vs. matched, actively infected cells. mRNA expression levels of all BTN3A isoforms (BTN3A1, BTN3A2, and BTN3A3) were elevated in CD4+ T cells from HIV infected individuals compared to a healthy donor.

### D. Pull Down Experiments to Verify Butyrophilin BTN3A as Biomarker on HIV Infected Cells.

Whole blood isolated from HIV-1+ individuals (on ART, viral loads<50 copies/ml) were procured from Hemacare Corporation (California). PBMCs were isolated following a Ficoll-Paque™ protocol. Total CD4+ T cells were isolated by negative selection using an EasySep™ Human CD4+ T Cell Isolation kit (Stemcell Technologies), as per manufacturer's instructions.

i. Immunoaffinity Enrichment with Antibody Beads:

Antibodies targeting BTN3A (clone 20.1, Biolegend, catalog #342702 and clone 103.2, Creative Biolabs, catalog #PABL-415); PD-1 (clone EH12.2H7, Biolegend, catalog #329902); or isotype control (clone P3.6.2.8.1, Thermo Fisher, catalog #16-4714-82) were conjugated using a Dynabeads™ Antibody Coupling Kit (Thermo Fisher, catalog #14311D), as per manufacturer's instructions. Antibody beads were then used for pull downs on CD4+ T cells isolated from HIV-1+ donors. Anti-BTN3A 20.1 is a pan anti-BTN3A antibody, which can not discriminate the three isoforms of BTN3A. See Vantourout et al., PNAS, 115(5):1039-1044, 2017.

ii. Analysis of HIV P24 and RNA Levels:

Following antibody pull downs, cells were treated with PMA (100 ng/ml)/Ionomycin (1 μg/ml) or DMSO (as control) in RPMI media containing 10% Fetal bovine serum (Gibco, Gaithersburg, Md.), penicillin (100U/ml)/streptomycin (0.1 mg/ml) (Gibco, Gaithersburg, Md.), and L-Glutamine (2 mM) (Gibco, Gaithersburg, Md.) for 24h. Cells and culture supernatants were collected at indicated time points for HIV P24 and RNA analyses.

a. HIV-1 P24 analysis: Cells and culture supernatants were lysed/inactivated in 1% Triton X 100. HIV P24 levels were measured using a Simoa HIV p24 immunoassay, as per manufacturer's instructions [3, 4].
b. HIV RNA analysis: Total RNA was isolated from cells using a RNAeasy kit (Qiagen) and cDNA synthesis was performed using a TaqMan Gene Expression Cells-to-CT Kit (Thermo fisher). ISCA was performed using a protocol based on [5].

### III. HIV DNA Analysis:

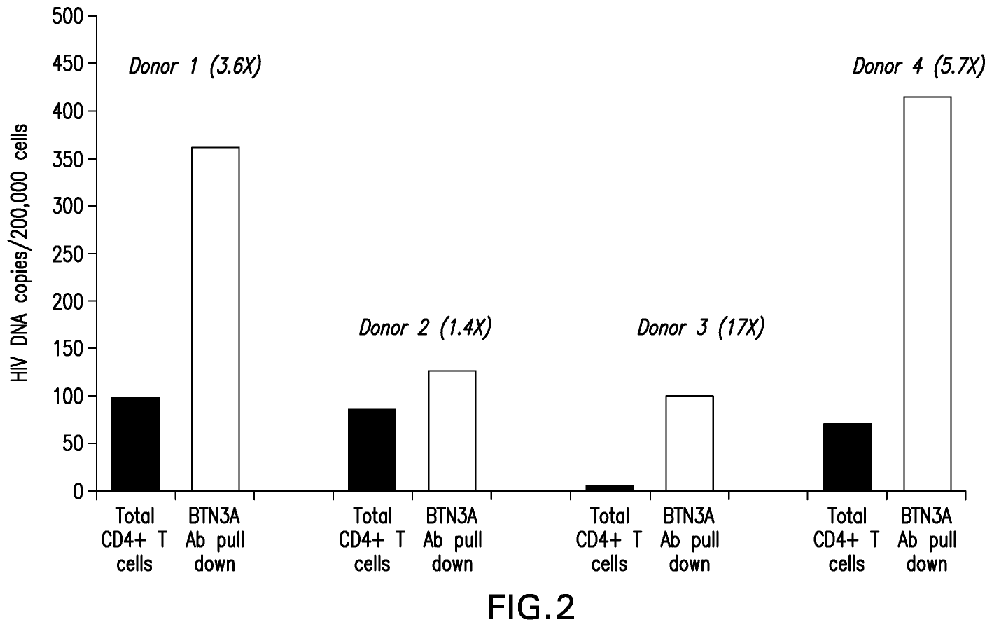

Following pull downs cells were lysed in proteinase K digestion buffer (10 mM Tris-HCl, pH 8.0, 50 nM KCl, 400 μg/ml proteinase K) for 16 h at 55° C. in a heating shaker. Proteinase K was inactivated by heating the digested samples at 95° C. for 5 min. Cell lysates were immediately used for HIV DNA quantification using a HIV LTR real-time PCR. DNA template preparation was based on the following manuscripts [6, 7]. Pulldown of CD4+ T-cells from HIV+ ART suppressed subjects with anti-BTN3 antibody shows enrichment of HIV-1 infected cells. HIV RNA (5 of 8 donors) and P24 (8 of 8 donors) protein levels are increased in antibody pull down cells as compared to total CD4+ T cells. Values range from 0.44- to 14-fold for HIV RNA and 1- to 36-fold for HIV P24. Further, HIV DNA (4 of 4 donors) is increased in antibody pull down cells as compared to total CD4+ T cells with values ranging from 1.4- to 17-fold over total CD4+ T cells (FIG. 2). Together, this data indicates that HIV infected cells demonstrate higher levels of BTN3 expression as compared to total CD4+ T cell populations.

### Example 2: Impact of BTN Modulation on T Cell Activation

### A. Inhibition of T Cell Responses by Human Butyrophilins:

To characterize the role of butyrophilins in regulating T cell responses, T cell assays were performed using recombinant human butyrophilin-Fc proteins. Specifically, we assessed the role of butyrophilins in inhibiting T cell receptor (TCR)-mediated T cell activation by anti-CD3 antibody (clone OKT3). Experiments were performed in bead- and plate-based formats.

i. Bead-Based Assay:

Briefly, PBMCs were isolated from healthy human donors (Biological Specialty Corporation, Colmar, Pa.). To generate T cell blasts, PBMCs were treated with IL-2 (4u/ml) and PHA (1 μg/ml) in RPMI media containing 10% Fetal bovine serum (Gibco, Gaithersburg, Md.), penicillin (100U/ml)/streptomycin (0.1 mg/ml) (Gibco, Gaithersburg, Md.), and L-Glutamine (2 mM) (Gibco, Gaithersburg, Md.) at 37° C. in a CO₂ incubator for 7 days. Fresh media was added once every 3 days.

Total CD4+ T cell populations were purified by negative selection using an EasySep™ Human CD4+ T Cell Enrichment Kit (Stemcell Technologies, Vancouver, Canada) as per manufacturer's instructions and resulted in ˜95% purity. Mouse anti-human CD3 antibody, control IgG, and fusion proteins (BTN2A2-Fc, BTN3A1-Fc, and PD-L1-Fc) were biotinyated using a Biotin type A Fast Conjugation kit (Abcam, Cambridge, United Kingdom). Biotinylated anti-CD3 antibody and fusion proteins were then attached to Dynabeads M280 streptavidin (Thermo Fisher Scientific, Waltham, Mass.) as per manufacturer's instructions at the following ratios:

1. Control IgG beads: anti CD3 antibody (30%), control IgG (70%)

2. BTN2A2-Fc beads: anti CD3 antibody (30%), BTN2A2-Fc (40%), control IgG (30%)

3. BTN3A1-Fc beads: anti CD3 antibody (30%), BTN3A1-Fc (40%), control IgG (30%)

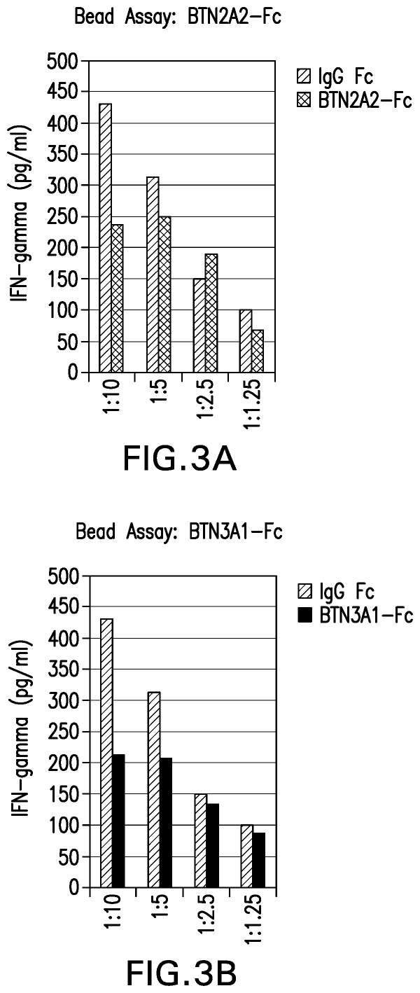

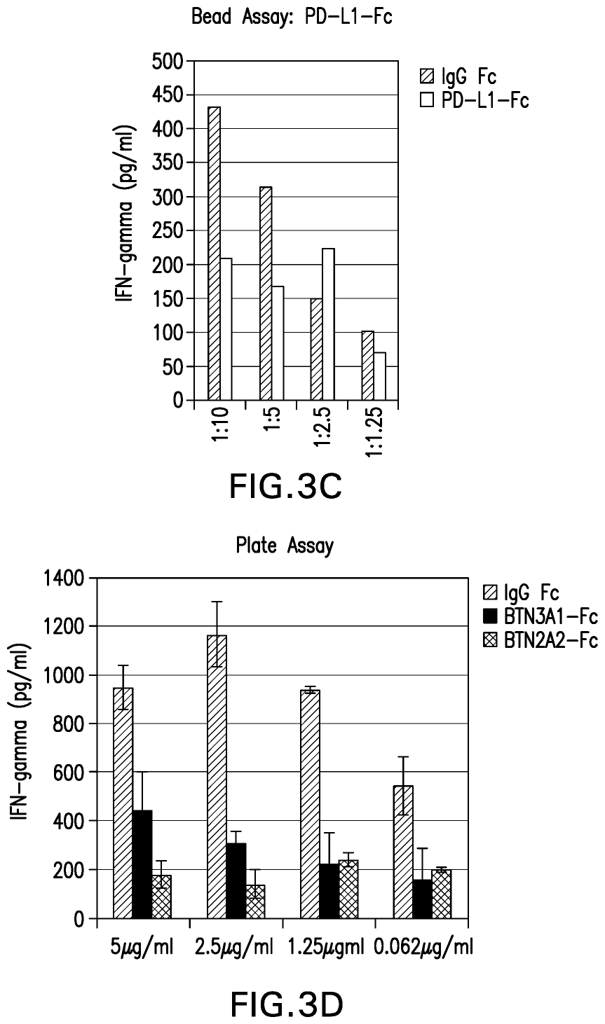

4. PD-L1-Fc beads: anti CD3 antibody (30%), PD-L1-Fc (40%), control IgG (30%). 2×10⁵ CD4+ T cells were seeded in 96-well flat-bottomed plates and beads were added at 1:10, 1:5, and 1:1.25 cell:bead ratios in RPMI media containing 10% Human serum (Sigma, H4522), penicillin (100U/ml)/streptomycin (0.1 mg/ml), and L-Glutamine (2 mM) and incubated at 37° C. in a CO₂ incubator for 96h. T cell activation was measured based on IFNγ expression using a V-PLEX proinflammatory panel 1 human assay (Meso Scale Discovery, Rockville, Md.) (FIG. 3A-C).

ii. Plate-Based Assay

Sterile 96-well flat bottom tissue culture plates (Corning) were coated with 1 μg/ml of anti-CD3 antibody (Clone OKT3, BD Pharmingen) in PBS+/− the following Fc-fusion proteins: BTN2A2-Fc, BTN3A1-Fc, or human IgG Fc. Coating was done in a two-step protocol. First, anti-CD3 antibody was added to the plates and incubated for 16 h in a 4° C. refrigerator. Next, recombinant BTNs or human IgG Fc proteins were diluted in PBS to 10, 5, 2.5, and 1.25 μg/ml and added at 100 ul/well. 10 ul/well of goat anti-human IgG Fc (R&D systems), diluted to 100 μg/ml in PBS was then added to each well and incubated for 3-4h at 37° C. Recombinant BTNs were procured from R&D systems as custom orders; human IgG Fc (catalog #110-HG-100). Total CD4+ T cells (prepared as described in section 1A) were then added to BTN-coated plates at 100,000 cells/well in RPMI media containing 10% Human serum (Sigma), penicillin (100 U/ml)/streptomycin (0.1 mg/ml), and L-Glutamine (2 mM) and incubated at 37° C. in a CO₂ incubator for 72 h. T cell activation was measured based on IFNγ expression using a V-PLEX proinflammatory panel 1 human assay (Meso Scale Discovery, Rockville, Md.) (FIG. 3D).

Recombinant BTN proteins, BTN2A2 and BTN3A1, and PD-L1 block activation of human CD4+ T cells based on IFNγ production (FIG. 3), T cell proliferation, and IL-2 production in both plate and bead-based assays. This is consistent with several previous reports for other B7 family members including, PD-L1. These results suggest a potential role for BTNs as immunomodulatory receptors.

### B. Activation of Human T Cell Responses by Anti-BTN3A Antibodies:

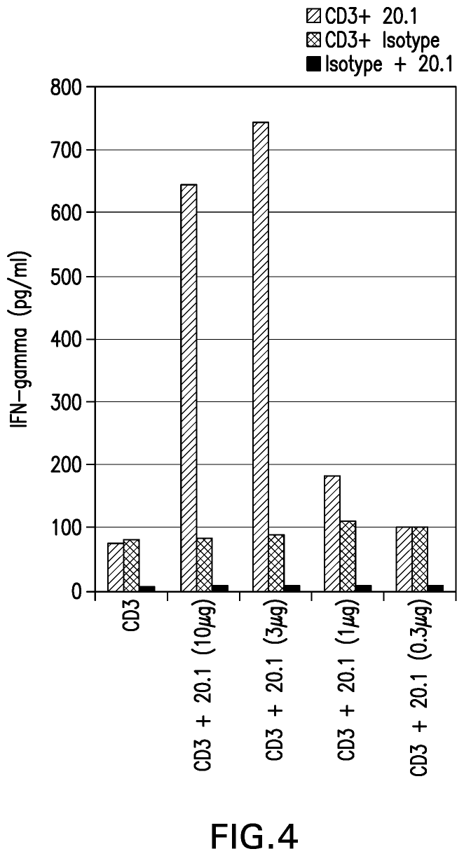

We next addressed if treatment with anti-BTN3A antibodies reverses BTN3A-mediated blockade in T cell activation. To assess this, sterile 96-well flat bottom tissue culture plates (Corning) were coated with various concentrations of anti-CD3 and anti-BTN3 20.1 antibodies (10 μg/ml, 3 μg/ml, 1 μg/ml, and 0.3 μg/ml) in PBS for 16 h in a 4° C. refrigerator. As controls, plates were coated with various concentrations of anti-CD3 antibody and isotype control antibody (10 μg/ml, 3 μg/ml, 1 μg/ml, and 0.3 μg/ml), anti-CD3 antibody alone, or isotype control antibody alone. The next day, antibodies were aspirated using a multichannel pipette and HIV latent primary CD4+ T cells from two donors were added to the antibody coated plates in duplicates at 125,000 cells/well in 200 ul of primary cell media (RPMI with 10% FBS, 50 μg/ml Primocin™). At 72 h following treatment T cell activation was measured based on IFNγ expression using a V-PLEX proinflammatory panel 1 human assay (Meso Scale Discovery, Rockville, Md.) (FIG. 4).

Treatment with anti-BTN3A antibody (clone 20.1) releases the BTN induced inhibitory blockade and activates human primary CD4+ T cells (based on IFNγ production, when used in combination with an anti-CD3 antibody (FIG. 4).

### Example 3: Impact of BTN Modulation on HIV Latency

A. HIV Latency Reversal Resulting from BTN Modulation

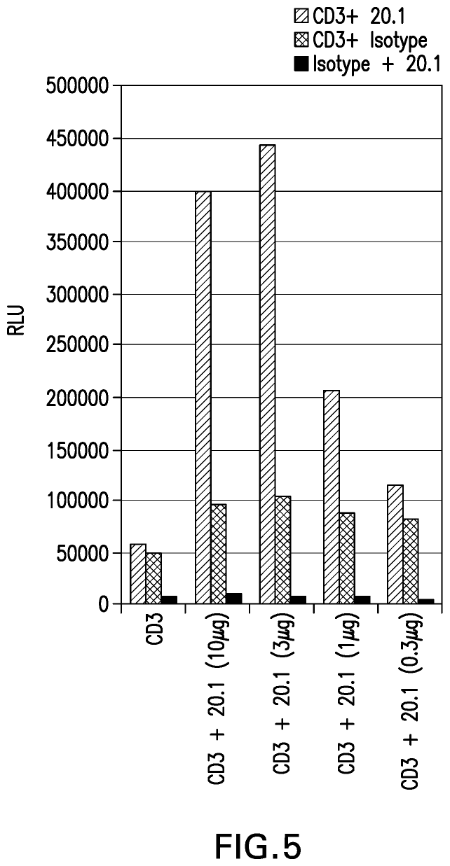

To assess if BTN3A modulation has impact on HIV latency reversal, sterile 96-well flat bottom tissue culture plates (Corning) were coated with various concentrations of anti-CD3 and anti-BTN3 20.1 antibodies (10 μg/ml, 3 μg/ml, 1 μg/ml, and 0.3 μg/ml) in PBS for 16h in a 4° C. refrigerator. As controls, plates were coated with various concentrations of anti-CD3 antibody and isotype control antibody (10 μg/ml, 3 μg/ml, 1 μg/ml, and 0.3 μg/ml), anti-CD3 antibody alone, or isotype control antibody alone. The next day, antibodies were aspirated using a multichannel pipette and HIV latent primary CD4+ T cells from two donors were added to the antibody coated plates in duplicates at 125,000 cells/well in 200 ul of primary cell media (RPMI with 10% FBS, 50 μg/ml Primocin™). At 24 h post treatment latent virus reactivation was measured using a Nano-Glo® Luciferase Assay (Promega) (FIG. 5).

Treatment of HIV latent primary CD4+ T cells with anti BTN3 20.1 antibody reactivates latent HIV when used in combination with anti-CD3 antibody (FIG. 5).

### B. Competition Experiments to Assess the Specificity of Anti-BTN3A-Mediated HIV Activity:

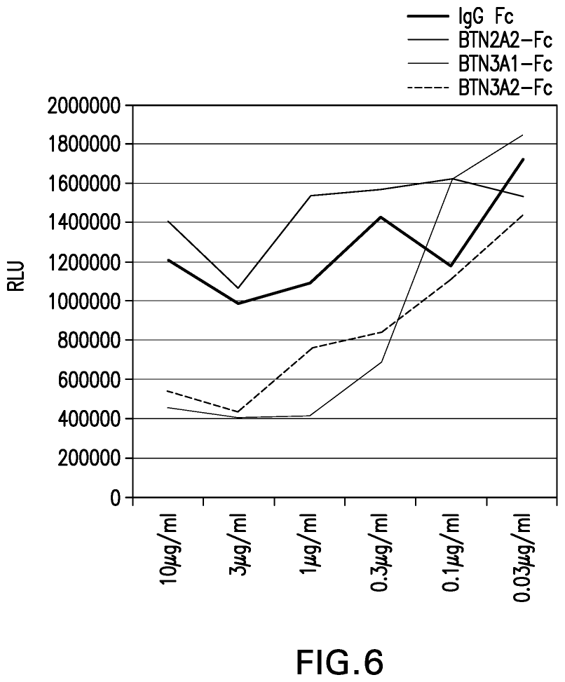

To confirm the specificity of anti-BTN3A antibody-mediated activity on latent virus activity and T cell activation, we performed competition experiments using recombinant BTN proteins. Briefly, 96-well flat bottom tissue culture plates (Corning) were coated with anti-CD3 (1 μg/ml) and anti-BTN3A 20.1 antibody (5 μg/ml) in PBS for 16 h in a 4° C. refrigerator. The next day, antibodies were aspirated using a multichannel pipette and various concentrations of recombinant BTN-Fc proteins BTN3A1-Fc and BTN3A2-Fc (10 μg/ml, 3 μg/ml, 1 μg/ml, 0.3 μg/ml, 0.1 μg/ml, and 0.03 μg/ml) were added to antibody coated plates in 2% FBS (in PBS). BTN2A2-Fc and human IgG Fc were used as controls. After 30 min incubation at RT recombinant Fc proteins were aspirated using a multichannel pipette. Next, HIV latent primary CD4+ T cells from two donors were added to the antibody coated plates in duplicates at 125,000 cells/well in 200 ul of primary cell media (RPMI with 10% FBS, 50 μg/ml Primocin™). At 24h post treatment virus reactivation was measured using a Nano-Glo® Luciferase Assay (Promega) (FIG. 6). A dose-dependent inhibition in anti-BTN3 20.1 Ab-mediated latent HIV activation and T cell activation was observed following treatment with BTN3A1-Fc and BTN3A2-Fc proteins, but not by BTN2A2-Fc or IgG Fc controls (FIG. 6).

### Example 4: Impact of BTN3A Antibodies on HIV-Gag Peptide Stimulated Lymphocytes

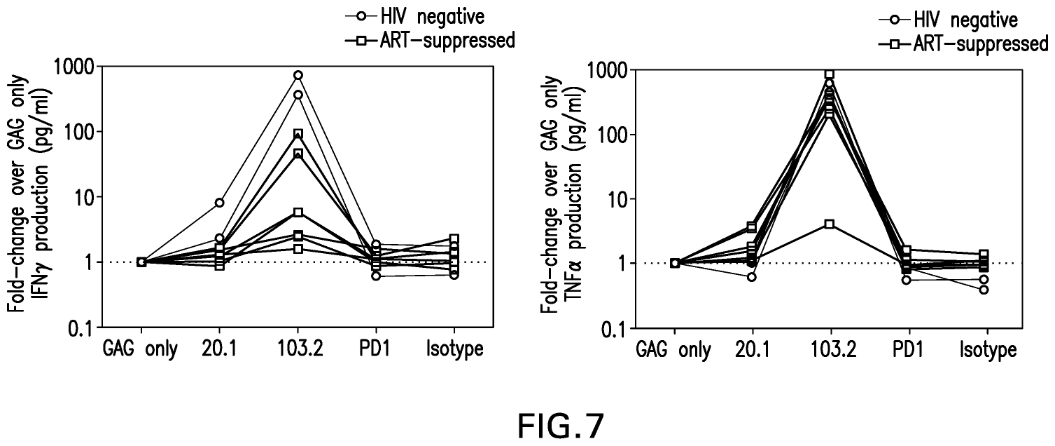

Thawed, PBMC isolated from samples of patients that are HIV-negative or HIV-positive on anti-retroviral therapy with undetectable viral load (“ART-suppressed”) were washed twice in 1×PBS. Following wash, PBMC were resuspended in medium (RPMI, supplemented with 10% FBS, and 1% Penicillin/Streptomycin) at a concentration of [1×10⁷ cells/mL]. 100 uL [1×10⁶ cells/well] of suspensions were aliquoted in per well of a 96-well round bottom plate (Corning). Plates were centrifuged and supernatants were decanted. Cells were resuspended with 100 uL of medium containing: either anti-BTN3A (clone 20.1, Biolegend, catalog #342702 or clone 103.2, Creative Biolabs, catalog #PABL-415), anti-PD-1 (in-house MK-3475) or isotype as controls [10 ug/mL, prepared in medium] or medium alone and incubated for 30 minutes prior to the addition of 100 uL of HIV-gag pool peptide [4 ug/mL prepared in medium]. Plates were incubated for 6 hours at 37° C. (5.0% CO₂). Following stimulation, plates were centrifuged and supernatants were removed and stored at −80° C. in 96-well U-bottom plates (Corning) until analysis. Activation was determined by expression of IFNγ and TNFα in the supernatants using a V-PLEX proinflammatory panel 1 human assay (Meso Scale Discovery, Rockville, Md.). The results were normalized as fold change in cytokine production relative to HIV-gag stimulation in the absence of antibody. Among the test articles, pre-treatment with anti-BTN3A (clone 103.2)-antibody markedly impacted activation resulting in upregulation of proinflammatory cytokines in the presence of HIV-gag pool peptide stimulation (FIG. 7).

### Example 5: Generation of Anti-BTN3A Antibodies and Screening for Functional Anti-BTN3A Antibodies

De novo antibody discovery for butyrophilin isoforms was executed on pre-immune yeast display libraries. The soluble proteins used in the yeast display selections were biotinylated recombinant proteins of HEK293-expressed Fc-chimeras BTN2A2, BTN3A1, BTN3A2, and BTN3A3 from R&D systems. All proteins were analyzed before use by Biacore binding against control antibody anti-BTN Monoclonal Antibody (eBioBT3.1 (20.1, BT3.1), SEC, SDS-PAGE, and endotoxin.

Each biotinylated BTN isoform was subjected to pre-immune yeast display libraries for selections. The selections started with MACS (Magnetic Bead-based Cell Separation) cell separations for the first two rounds to enrich clones that bind to the targeted BTN isoforms, and applied fluorescence activated cell sorting (FACS) at later rounds to isolate clones that were with high affinity and isoform specificity. Due to the high sequence homology among all BTN isoforms, isoform-specific selection pressures were achieved by introducing ten-fold molar ratio of the non-biotinylated BTN isoforms to the biotinylated and selection-targeted BTN isoforms in the later rounds of the selections.

The isolated clones were then sequenced to identify the unique antibodies and screened for isoform binding profiles by Octet. To ensure the isolated antibodies have broad-epitopic coverage, all the antibodies were subjected to binning against the benchmark antibodies: BTN20.1 (Biolegend, agonist) and BTN103.2 (Creative Biolabs, antagonist). Purified antibodies were submitted for functional assay screening, as described below.

As demonstrated in the associated tables, antibodies generated above exhibit a mixture of both specific and cross-reactive binding to BTN3A isoforms but did not bind to BTN2A2 (Table 3). Given the sequence homology between BTN3A isoforms (>90%), this outcome is expected. Within all 105 antibodies from the BTN3A1 campaign and 125 antibodies from the BTN3A2 campaign, each antibody was characterized into one of the bins: Biolegend-bin, Creative-Biolabs-bin, Empty Set, or Overlapping.

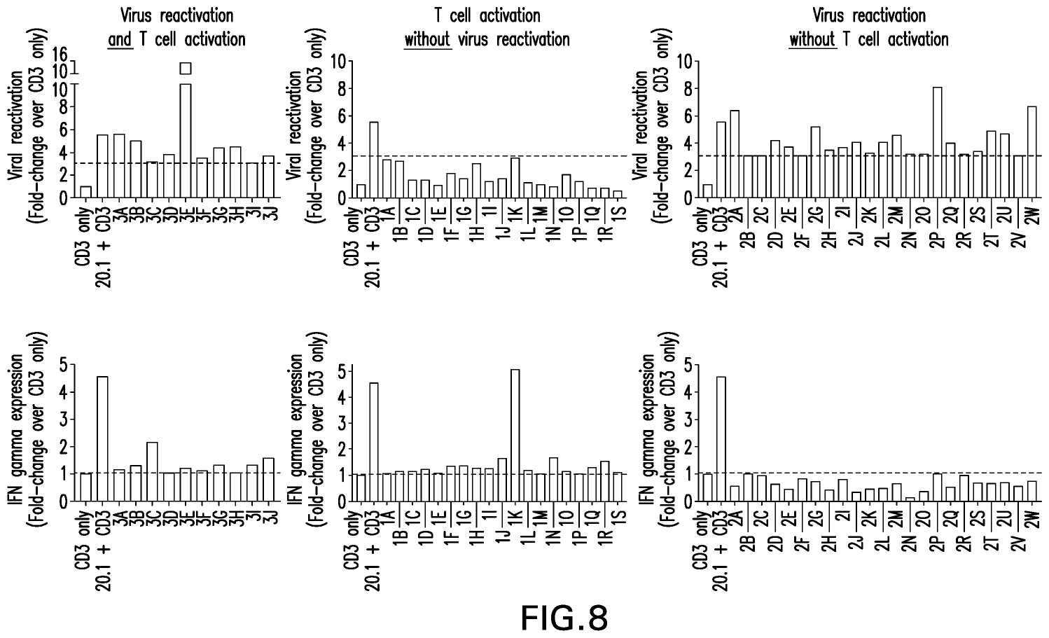

Isoform binding specificity and cross-reactivity for antibodies with a positive functional profile are summarized in Table 3. Functional antibodies were defined by the ability to activate T cells and/or reactivate latent HIV. Detailed functional results can be found in FIG. 8.

TABLE 3
Binding and functional characterization of
human IgG yeast display library antibodies.

| | | | | | Antibody function | |
|---|---|---|---|---|---|---|
| | BTN isoform binding | | | | T cell | HIV |
| Antibody ID | 3A1 | 3A2 | 3A3 | 2A2 | activation | reactivation |
| 1A | + | + | + | − | Yes | No |
| 1B | + | − | + | − | Yes | No |
| 1C | + | + | + | − | Yes | No |
| 1D | + | + | + | − | Yes | No |
| 1E | + | − | + | − | Yes | No |
| 1F | + | − | − | − | Yes | No |
| 1G | + | + | + | − | Yes | No |
| 1H | + | − | + | − | Yes | No |
| 1I | + | − | − | − | Yes | No |
| 1J | + | + | − | − | Yes | No |
| 1K | − | − | − | − | Yes | No |
| 1L | + | + | − | − | Yes | No |
| 1M | + | + | + | − | Yes | No |
| 1N | + | + | − | − | Yes | No |
| 1O | + | + | − | − | Yes | No |
| 1P | + | + | + | − | Yes | No |
| 1Q | − | − | − | − | Yes | No |
| 1R | − | + | + | − | Yes | No |
| 1S | + | + | + | − | Yes | No |
| 2A | + | − | + | − | No | Yes |
| 2B | + | + | + | − | No | Yes |
| 2C | + | + | + | − | No | Yes |
| 2D | + | + | + | − | No | Yes |
| 2E | + | + | + | − | No | Yes |
| 2F | + | − | − | − | No | Yes |
| 2G | + | − | + | − | No | Yes |
| 2H | + | + | + | − | No | Yes |
| 2I | + | + | + | − | No | Yes |
| 2J | + | − | + | − | No | Yes |
| 2K | + | − | + | − | No | Yes |
| 2L | + | − | − | − | No | Yes |
| 2M | + | − | − | − | No | Yes |
| 2N | + | − | − | − | No | Yes |
| 2O | + | − | − | − | No | Yes |
| 2P | + | + | + | − | No | Yes |
| 2Q | + | + | + | − | No | Yes |
| 2R | + | + | − | − | No | Yes |
| 2S | + | + | − | − | No | Yes |
| 2T | − | − | − | − | No | Yes |
| 2U | + | − | − | − | No | Yes |
| 2V | + | + | − | − | No | Yes |
| 2W | + | + | + | − | No | Yes |
| 3A | + | − | + | − | Yes | Yes |
| 3B | + | + | + | − | Yes | Yes |
| 3C | + | + | + | − | Yes | Yes |
| 3D | + | + | + | − | Yes | Yes |
| 3E | + | + | − | − | Yes | Yes |
| 3F | + | + | + | − | Yes | Yes |
| 3G | − | − | − | − | Yes | Yes |
| 3H | + | + | + | − | Yes | Yes |
| 3I | + | + | − | − | Yes | Yes |
| 3J | + | + | + | − | Yes | Yes |

Using the protocols described in detail in Example 2B and Example 3A, anti-BTN3A antibodies were screened for a functional role in T cell activation and reactivation of latent HIV. Antibody screens were conducted using HIV latent CD4+ T cells (prepared as described in Example 1A). T cell activation was quantified by production of IFNγ, and reactivation of latent HIV was quantified by luciferase assay. FIG. 8 shows compiled functional data for all antibody hits from the BTN3A1 campaign. Detailed information on binding profile with binding affinity (KD) (Table 4) and antibody isotype and sequence (Table 5) are provided for two representative antibodies from each of the three functional categories observed.

Hits from this screen fall into three functional categories: T cell activation without virus reactivation (19 hits); Virus reactivation without T cell activation (23 hits); and Virus reactivation and T cell activation (10 hits). There was no statistical correlation between BTN3A binding specificity and functional profile. Table 4 provides the antibody binding profile, binding affinity of two representative antibodies from each of the three functional categories (IgG1N297A isotype).

TABLE 4
Antibody binding profile: isoform-specific binding and binding affinity (KD)

| | BTN3A1 | | BTN3A2 | | BTN3A3 | | |
|---|---|---|---|---|---|---|---|
| Antibody ID | Binding | KD | Binding | KD | Binding | KD | Isotype |
| 1F | Yes | 1.531E−09 | No | n.a. | No | n.a. | hIgG1-297A |
| 1G | Yes | 9.742E−10 | Yes | 2.707E−09 | Yes | 1.133E−09 | hIgG1-297A |
| 2A | Yes | 4.381E−09 | No | n.a. | Yes | 8.524E−09 | hIgG1-297A |
| 2L | Yes | 3.941E−09 | No | n.a. | No | n.a. | hIgG1-297A |
| 3A | Yes | 2.757E−09 | No | n.a. | Yes | 1.402E−09 | hIgG1-297A |
| 3B | Yes | 2.104E−09 | Yes | 4.568E−09 | Yes | 2.085E−09 | hIgG1-297A |

TABLE 5
Antibody heavy and light chain variable region sequences.

| | | | Light | |
|---|---|---|---|---|
| Antibody | Heavy chain | | chain | |
| ID | germline | Heavy chain sequence | germline | Light chain sequence |
| 1F | VH4-31.5 | QVQLQESGPGLVKPSQ | VK3-20.0 | EIVLTQSPGTLSLSPG |
| | | TLSLTCTVSGGSISSGG | | ERATLSCRASQSVSS |
| | | YYWSWIRQHPGKGLE | | SYLAWYQQKPGQAP |
| | | WIGSIYYSGSTYYNPSL | | RLLIYGASSRATGIPD |
| | | KSRVTISVDTSKNQFSL | | RFSGSGSGTDFTLTIS |
| | | KLSSVTAADTAVYYCA | | RLEPEDFAVYYCQQ |
| | | RLHSSQSSSTYWGQGT | | YGHYPYTFGGGTKV |
| | | LVTVSS (SEQ ID NO: 1) | | EIK (SEQ ID NO: 2) |
| 1G | VH1-18.0 | QVQLVQSGAEVKKPGA | VK3- | EIVLTQSPATLSVSPG |
| | | SVKVSCKASGYTFTSY | 15.10 | ERATLSCRASQSVSS |
| | | GISWVRQAPGQGLEW | | NLAWYQQKPGQAPR |
| | | MGWISAYNGNTNYAQ | | LLIYGASTRATGIPA |
| | | KLQGRVTMTTDTSTST | | RFSGSGSGTEFTLTIS |
| | | AYMELRSLRSDDTAVY | | SLQSEDFAVYYCQQ |
| | | YCARLGATVAYFDLW | | DVYWPFTFGGGTKV |
| | | GRGTLVTVSS (SEQ ID | | EIK (SEQ ID NO: 4) |
| | | NO: 3) | | |
| 2A | VH1-46.4 | QVQLVQSGAEVKKPGA | VK3-15.1 | EIVMTQSPATLSVSP |
| | | SVKVSCKASGYTFTSY | | GERATLSCRASQSVG |
| | | YIHWVRQAPGQGLEW | | SNLAWYQQKPGQAP |
| | | MGIINPSGGSTSYAQKF | | RLLIYGASTRATGIP |
| | | QGRVTMTRDTSTSTVY | | ARFSGSGSGTEFTLTI |
| | | MELSSLRSEDTAVYYC | | SSLQSEDFAVYYCQ |
| | | ARATWEALHYWGQGT | | QYYAWPRTFGGGTK |
| | | LVTVSS (SEQ ID NO: 5) | | VEIK (SEQ ID NO: 6) |
| 2L | VH1-02.6 | QVQLVQSGAEVKKPGA | VK1-12.7 | DIQMTQSPSSVSASV |
| | | SVKVSCKASGYTFTGY | | GDRVTITCRASQGIS |
| | | YMHWVRQAPGQGLEW | | SWLAWYQQKPGKA |
| | | MGSINPNSGGTNYAQK | | PKLLIYAASNLQSGV |
| | | FQGRVTMTRDTSISTAY | | PSRFSGSGSGTDFTL |
| | | MELSRLRSDDTAVYYC | | TISSLQPEDFATYYC |
| | | ARPPEVVGYGEDWFDP | | QQGNDLPITFGGGTK |
| | | WGQGTLVTVSS (SEQ | | VEIK (SEQ ID NO: 8) |
| | | ID NO: 7) | | |
| 3A | VH1-46.8 | QVQLVQSGAEVKKPGA | VK1-05.6 | DIQMTQSPSTLSASV |
| | | SVKVSCKASGYTFTSY | | GDRVTITCRASQSISS |
| | | YMHWVRQAPGQGLEW | | WLAWYQQKPGKAP |
| | | MGIINPGGGSTSYAQKF | | KLLIYKASSLESGVP |
| | | QGRVTMTRDTSTSTVY | | SRFSGSGSGTEFTLTI |
| | | MELSSLRSEDTAVYYC | | SSLQPDDFATYYCQ |
| | | ARDAGHDYGDMAYW | | QYRSFPTFGGGTKVE |
| | | GQGTLVTVSS (SEQ ID | | IK (SEQ ID NO: 10) |
| | | NO: 9) | | |
| 3B | VH1-46.0 | QVQLVQSGAEVKKPGA | VK3-11.0 | EIVLTQSPATLSLSPG |
| | | SVKVSCKASGYTFTSY | | ERATLSCRASQSVSS |
| | | YMHWVRQAPGQGLEW | | YLAWYQQKPGQAPR |
| | | MGIINPSGGSTSYAQKF | | LLIYDASNRATGIPA |
| | | QGRVTMTRDTSTSTVY | | RFSGSGSGTDFTLTIS |
| | | MELSSLRSEDTAVYYC | | SLEPEDFAVYYCQQ |
| | | ARPSGDYSGYDALDV | | DPYWPITFGGGTKVE |
| | | WGQGTMVTVSS (SEQ | | IK (SEQ ID NO: 12) |
| | | ID NO: 11) | | |

CDR regions are underlined and were identified by Kabat numbering system.

TABLE 6
Antibody heavy and light chain CDR sequences.

| Antibody | | |
|---|---|---|
| ID | Heavy chain CDR sequences | Light chain CDR sequences |
| 1F | CDRH1: GSISSGGYYWS (SEQ | CDRL1: RASQSVSSSYLA (SEQ ID |
| | ID NO: 13) | NO: 16) |
| | CDRH2: SIYYSGSTYYNPSLKS | CDRL2: GASSRAT (SEQ ID NO: 17) |
| | (SEQ ID NO: 14) | CDRL3: QQYGHYPYT (SEQ ID NO: |
| | CDRH3: ARLHSSQSSSTY (SEQ | 18) |
| | ID NO: 15) | |
| 1G | CDRH1: YTFTSYGIS (SEQ ID | CDRL1: RASQSVSSNLA (SEQ ID NO: |
| | NO: 19) | 22) |
| | CDRH2: WISAYNGNTNYAQKLQ | CDRL2: GASTRAT (SEQ ID NO: 23) |
| | G (SEQ ID NO: 20) | CDRL3: QQDVYWPFT (SEQ ID NO: |
| | CDRH3: ARLGATVAYFDL (SEQ | 24) |
| | ID NO: 21) | |
| 2A | CDRH1: YTFTSYYIH (SEQ ID | CDRL1: RASQSVGSNLA (SEQ ID |
| | NO: 25) | NO: 28) |
| | CDRH2: IINPSGGSTSYAQKFQG | CDRL2: GASTRAT (SEQ ID NO: 29) |
| | (SEQ ID NO: 26) | CDRL3: QQYYAWPRT (SEQ ID NO: |
| | CDRH3: ARATWEALHY (SEQ ID | 30) |
| | NO: 27) | |
| 2L | CDRH1: YTFTGYYMH (SEQ ID | CDRL1: RASQGISSWLA (SEQ ID NO: |
| | NO: 31) | 34) |
| | CDRH2: SINPNSGGTNYAQKFQ | CDRL2: AASNLQS (SEQ ID NO: 35) |
| | G (SEQ ID NO: 32) | CDRL3: QQGNDLPIT (SEQ ID NO: |
| | CDH3: ARPPEVVGYGEDWFDP | 36) |
| | (SEQ ID NO: 33) | |
| 3A | CDRH1: YTFTSYYMH (SEQ ID | CDRL1: RASQSISSWLA (SEQ ID NO: |
| | NO: 37) | 40) |
| | CDRH2: IINPGGGSTSYAQKFQG | CDRL2: KASSLES (SEQ ID NO: 41) |
| | (SEQ ID NO: 38) | CDRL3: QQYRSFPT (SEQ ID NO: 42) |
| | CDRH3: ARDAGHDYGDMAY (SEQ | |
| | ID NO: 39) | |
| 3B | CDRH1: YTFTSYYMH (SEQ ID | CDRL1: RASQSVSSYLA (SEQ ID NO: |
| | NO: 43) | 46) |
| | CDRH2: IINPSGGSTSYAQKFQG | CDRL2: DASNRAT (SEQ ID NO: 47) |
| | (SEQ ID NO: 44) | CDRL3: QQDPYWPIT (SEQ ID NO: |
| | CDRH3: ARPSGDYSGYDALDV | 48) |
| | (SEQ ID NO: 45) | |

### REFERENCES

1. Arnett H A, Viney J L. Immune modulation by butyrophilins. Nature reviews. 14(8):559-69. PubMed PMID: 25060581.
2. Nguyen K, Das B, Dobrowolski C, Karn J. Multiple Histone Lysine Methyltransferases Are Required for the Establishment and Maintenance of HIV-1 Latency. mBio. 8(1). PubMed PMID: 28246360.
3. Passaes C P, Bruel T, Decalf J, David A, Angin M, Monceaux V, et al. Ultrasensitive HIV-1 p24 Assay Detects Single Infected Cells and Differences in Reservoir Induction by Latency Reversal Agents. Journal of virology. 91(6). PubMed PMID: 28077644.
4. Wu G, Swanson M, Talla A, Graham D, Strizki J, Gorman D, et al. HDAC inhibition induces HIV-1 protein and enables immune-based clearance following latency reversal. JCI insight. 2(16). PubMed PMID: 28814661.
5. Cillo A R, Vagratian D, Bedison M A, Anderson E M, Kearney M F, Fyne E, et al. Improved single-copy assays for quantification of persistent HIV-1 viremia in patients on suppressive antiretroviral therapy. Journal of clinical microbiology. 52(11):3944-51. PubMed PMID: 25187636.
6. Abdel-Mohsen M, Kuri-Cervantes L, Grau-Exposito J, Spivak A M, Nell R A, Tomescu C, et al. CD32 is expressed on cells with transcriptionally active HIV but does not enrich for HIV DNA in resting T cells. Science translational medicine. 10(437). PubMed PMID: 29669853.
7. Vandergeeten C, Fromentin R, Merlini E, Lawani M B, DaFonseca S, Bakeman W, et al. Cross-clade ultrasensitive PCR-based assays to measure HIV persistence in large-cohort studies. Journal of virology. 88(21):12385-96. PubMed PMID: 25122785.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

1. A method of treating HIV in a subject comprising the step of administering to the subject a therapeutically effective amount of an antagonist anti-Butyrophilin3A (BTN3A) antibody, wherein the antibody activates CD4⁺ T cells and reactivates HIV from latency.

2. The method of claim 1, wherein the antibody increases IFNγ production, IL-2 production or T-cell proliferation in HIV latent primary CD4+ T cells and upregulates HIV transcription in CD4+ T cells.

3. The method of claim 1, wherein the antibody cross competes with anti-BTN3A antibody BTN20.1 or BTN103.2.

4. A method of reactivating HIV from latency in a subject comprising the step of administering to the subject a therapeutically effective amount of an antagonist anti-Butyrophilin3A (BTN3A) antibody, wherein the antibody reactivates HIV from latency.

5. The method of claim 4, wherein the antibody upregulates HIV transcription in CD4+ T cells.

6. A method of treating HIV in a subject, said method comprising the step of administering to the subject a therapeutically effective amount of an antagonist anti-Butyrophilin3A (BTN3A) antibody.

7. The method of claim 6, wherein the antibody kills HIV infected T cells.

8. A method of treating HIV in a subject comprising the step of administering to the subject a therapeutically effective amount of an antagonist anti-Butyrophilin3A (BTN3A) antibody, wherein the antibody activates CD4+ T cells.

9. The method of claim 8, wherein the antibody increases IFNγ production or T-cell proliferation in HIV latent primary CD4+ T cells.

10. The method of claim 1, wherein the anti-BTN3A antibody specifically binds to BTN3A1.

11. The method of claim 1, further comprising administering an anti-retroviral agent, wherein said anti-retroviral agent is selected from the group consisting of a nucleoside reverse transcriptase inhibitor, non-nucleoside reverse transcriptase inhibitor, protease inhibitor, fusion inhibitor, entry inhibitor, integrase inhibitor, co-receptor antagonist, viral adsorption inhibitor, viral specific transcription inhibitor, and cyclin dependent kinase inhibitor, or a combination thereof.

12. The method of claim 11, wherein said anti-retroviral agent is selected from the group consisting of a non-nucleoside reverse transcriptase inhibitor, a protease inhibitor, and an integrase inhibitor, or a combination thereof.

13. The method of claim 1, further comprising administering a latency reversing agent.


SEQUENCE LISTING (48 sequences)

SEQ ID NO: 1 - protein, 120 aa, Artificial Sequence
  Artificial polypeptide
QVQLQESGPGLVKPSQTLSLTCTVSGGSISSGGYYWSWIRQHPGKGLEWIGSIYYSGSTYYNPSLKSRVTISVDTSKNQFSLKLSSVTAADTAVYYCARLHSSQSSSTYWGQGTLVTVSS

SEQ ID NO: 2 - protein, 108 aa, Artificial Sequence
  Artificial polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGHYPYTFGGGTKVEIK

SEQ ID NO: 3 - protein, 119 aa, Artificial Sequence
  Artificial polypeptide
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARLGATVAYFDLWGRGTLVTVSS

SEQ ID NO: 4 - protein, 107 aa, Artificial Sequence
  Artificial polypeptide
EIVLTQSPATLSVSPGERATLSCRASQSVSSNLAWYQQKPGQAPRLLIYGASTRATGIPARFSGSGSGTEFTLTISSLQSEDFAVYYCQQDVYWPFTFGGGTKVEIK

SEQ ID NO: 5 - protein, 117 aa, Artificial Sequence
  Artificial polypeptide
QVQLVQSGAEVKKPGASVKVSCKASGYTFTSYYIHWVRQAPGQGLEWMGIINPSGGSTSYAQKFQGRVTMTRDTSTSTVYMELSSLRSEDTAVYYCARATWEALHYWGQGTLVTVSS

SEQ ID NO: 6 - protein, 107 aa, Artificial Sequence
  Artificial polypeptide
EIVMTQSPATLSVSPGERATLSCRASQSVGSNLAWYQQKPGQAPRLLIYGASTRATGIPARFSGSGSGTEFTLTISSLQSEDFAVYYCQQYYAWPRTFGGGTKVEIK

SEQ ID NO: 7 - protein, 123 aa, Artificial Sequence
  Artificial polypeptide
QVQLVQSGAEVKKPGASVKVSCKASGYTFTGYYMHWVRQAPGQGLEWMGSINPNSGGTNYAQKFQGRVTMTRDTSISTAYMELSRLRSDDTAVYYCARPPEVVGYGEDWFDPWGQGTLVTVSS

SEQ ID NO: 8 - protein, 107 aa, Artificial Sequence
  Artificial polypeptide
DIQMTQSPSSVSASVGDRVTITCRASQGISSWLAWYQQKPGKAPKLLIYAASNLQSGVPSRFSGSGSGTDFTLTISSLQPEDFATYYCQQGNDLPITFGGGTKVEIK

SEQ ID NO: 9 - protein, 120 aa, Artificial Sequence
  Artificial polypeptide
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARDAGHDYGDMAYWGQGTLVTVSS

SEQ ID NO: 10 - protein, 106 aa, Artificial Sequence
  Artificial polypeptide
DIQMTQSPSTLSASVGDRVTITCRASQSISSWLAWYQQKPGKAPKLLIYKASSLESGVPSRFSGSGSGTEFTLTISSLQPDDFATYYCQQYRSFPTFGGGTKVEIK

SEQ ID NO: 11 - protein, 122 aa, Artificial Sequence
  Artificial polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARPSGDYSGYDALDVWGQGTMVTVSS

SEQ ID NO: 12 - protein, 107 aa, Artificial Sequence
  Artificial polypeptide
EIVLTQSPATLSLSPGERATLSCRASQSVSSYLAWYQQKPGQAPRLLIYDASNRATGIPARFSGSGSGTDFTLTISSLEPEDFAVYYCQQDPYWPITFGGGTKVEIK

SEQ ID NO: 13 - protein, 11 aa, Artificial Sequence
  Artificial polypeptide
GSISSGGYYWS

SEQ ID NO: 14 - protein, 16 aa, Artificial Sequence
  Artificial polypeptide
SIYYSGSTYYNPSLKS

SEQ ID NO: 15 - protein, 12 aa, Artificial Sequence
  Artificial polypeptide
ARLHSSQSSSTY

SEQ ID NO: 16 - protein, 12 aa, Artificial Sequence
  Artificial polypeptide
RASQSVSSSYLA

SEQ ID NO: 17 - protein, 7 aa, Artificial Sequence
  Artificial polypeptide
GASSRAT

SEQ ID NO: 18 - protein, 9 aa, Artificial Sequence
  Artificial polypeptide
QQYGHYPYT

SEQ ID NO: 19 - protein, 9 aa, Artificial Sequence
  Artificial polypeptide
YTFTSYGIS

SEQ ID NO: 20 - protein, 17 aa, Artificial Sequence
  Artificial polypeptide
WISAYNGNTNYAQKLQG

SEQ ID NO: 21 - protein, 12 aa, Artificial Sequence
  Artificial polypeptide
ARLGATVAYFDL

SEQ ID NO: 22 - protein, 11 aa, Artificial Sequence
  Artificial polypeptide
RASQSVSSNLA

SEQ ID NO: 23 - protein, 7 aa, Artificial Sequence
  Artificial polypeptide
GASTRAT

SEQ ID NO: 24 - protein, 9 aa, Artificial Sequence
  Artificial polypeptide
QQDVYWPFT

SEQ ID NO: 25 - protein, 9 aa, Artificial Sequence
  Artificial polypeptide
YTFTSYYIH

SEQ ID NO: 26 - protein, 17 aa, Artificial Sequence
  Artificial polypeptide
IINPSGGSTSYAQKFQG

SEQ ID NO: 27 - protein, 10 aa, Artificial Sequence
  Artificial polypeptide
ARATWEALHY

SEQ ID NO: 28 - protein, 11 aa, Artificial Sequence
  Artificial polypeptide
RASQSVGSNLA

SEQ ID NO: 29 - protein, 7 aa, Artificial Sequence
  Artificial polypeptide
GASTRAT

SEQ ID NO: 30 - protein, 9 aa, Artificial Sequence
  Artificial polypeptide
QQYYAWPRT

SEQ ID NO: 31 - protein, 9 aa, Artificial Sequence
  Artificial polypeptide
YTFTGYYMH

SEQ ID NO: 32 - protein, 17 aa, Artificial Sequence
  Artificial polypeptide
SINPNSGGTNYAQKFQG

SEQ ID NO: 33 - protein, 16 aa, Artificial Sequence
  Artificial polypeptide
ARPPEVVGYGEDWFDP

SEQ ID NO: 34 - protein, 11 aa, Artificial Sequence
  Artificial polypeptide
RASQGISSWLA

SEQ ID NO: 35 - protein, 7 aa, Artificial Sequence
  Artificial polypeptide
AASNLQS

SEQ ID NO: 36 - protein, 9 aa, Artificial Sequence
  Artificial polypeptide
QQGNDLPIT

SEQ ID NO: 37 - protein, 9 aa, Artificial Sequence
  Artificial polypeptide
YTFTSYYMH

SEQ ID NO: 38 - protein, 17 aa, Artificial Sequence
  Artificial polypeptide
IINPGGGSTSYAQKFQG

SEQ ID NO: 39 - protein, 13 aa, Artificial Sequence
  Artificial polypeptide
ARDAGHDYGDMAY

SEQ ID NO: 40 - protein, 11 aa, Artificial Sequence
  Artificial polypeptide
RASQSISSWLA

SEQ ID NO: 41 - protein, 7 aa, Artificial Sequence
  Artificial polypeptide
KASSLES

SEQ ID NO: 42 - protein, 8 aa, Artificial Sequence
  Artificial polypeptide
QQYRSFPT

SEQ ID NO: 43 - protein, 9 aa, Artificial Sequence
  Artificial polypeptide
YTFTSYYMH

SEQ ID NO: 44 - protein, 17 aa, Artificial Sequence
  Artificial polypeptide
IINPSGGSTSYAQKFQG

SEQ ID NO: 45 - protein, 15 aa, Artificial Sequence
  Artificial polypeptide
ARPSGDYSGYDALDV

SEQ ID NO: 46 - protein, 11 aa, Artificial Sequence
  Artificial polypeptide
RASQSVSSYLA

SEQ ID NO: 47 - protein, 7 aa, Artificial Sequence
  Artificial polypeptide
DASNRAT

SEQ ID NO: 48 - protein, 9 aa, Artificial Sequence
  Artificial polypeptide
QQDPYWPIT